(19) United States
(12) Patent Application Publication
(10) Pub. No.: US 2025/0228672 A1
(43) Pub. Date: Jul. 17, 2025

(54) MODULAR MEDIAL PIVOT TIBIAL INSERT FOR ENDOPROSTHETIC KNEE IMPLANT ASSEMBLY

(71) Applicant: MicroPort Orthopedics Holdings Inc., Arlington, TN (US)

(72) Inventors: Brian R. Harris, JR., Cordova, TN (US); Paul S. Bryant, Collierville, TN (US); C. Brian McDaniel, Arlington, TN (US); Fred W. Bowman, Germantown, TN (US); Michael L. Brooks, Brighton, TN (US); Duy Q. Dang, Olive Branch, MS (US)

(73) Assignee: MicroPort Orthopedics Holdings Inc., Arlington, TN (US)

(21) Appl. No.: 19/016,037

(22) Filed: Jan. 10, 2025

Related U.S. Application Data
(60) Provisional application No. 63/620,761, filed on Jan. 12, 2024.
(60) Provisional application No. 63/552,096, filed on Feb. 10, 2024.

(51) Int. Cl. A61F 2/38; A61F 2/30

(52) U.S. Cl. CPC A61F 2/389; A61F 2/30734; A61F 2002/30153; A61F 2002/30331; A61F 2002/30387; A61F 2002/30476; A61F 2002/30604; A61F 2002/30624

(57) ABSTRACT

Exemplary assemblies, apparatuses, systems, methods, and kits are provided herein related to modular tibial inserts. One such assembly can comprise a modular tibial insert assembly having: a modular medial insert component, the modular medial insert component having a medial component proximal side proximally disposed from a medial component distal base side, wherein a medial component body extends between the medial component proximal side and the medial component distal base side; a modular lateral insert component, the modular lateral insert component having a lateral component proximal side proximally disposed from a lateral component distal base side, wherein a lateral component body extends between the lateral component proximal side and the lateral component distal base side; wherein the modular tibial insert assembly has an assembled configuration and a disassembled configuration, and wherein the modular medial insert component is configured to fixedly engage the modular lateral insert component in the assembled configuration.

### 1. RELATED APPLICATION

This application claims the benefit of U.S. Provisional Patent Application No. 63/620,761, filed on Jan. 12, 2024 and the benefit of U.S. Provisional Patent Application No. 63/552,096, filed on Feb. 10, 2024. The disclosures of these related priority applications are hereby incorporated into the present disclosure in their entireties.

### BACKGROUND OF THE INVENTION

### 2. TECHNICAL FIELD

The present disclosure relates generally to the field of orthopedic surgery, and more particularly to assemblies, apparatuses, systems, methods, and kits related to endoprosthetic knee implants.

### 3. RELATED ART

Endoprosthetic total knee implants are typically designed to abut resected surfaces of the patient's distal femur and proximal tibia. Because the surgeon is replacing sections of worn or damaged bone and soft tissue with artificial, biocompatible medical implant components, the surgeon is necessarily reconstructing a joint line for the patient.

Endoprosthetic total knee implants generally comprise a femoral component configured to be surgically implanted on and into a patient's resected distal femur, a tibial component configured to be surgically implanted on and into a patient's resected proximal tibia, and a tibial insert placed between the femoral and tibial components. The tibial insert generally serves as a bearing and permits the femoral component to rotate around the articular surface of the tibial insert as the patient bends and flexes the patient's knee.

The position of the patient's post-operative joint line depends on many factors including, but not limited to, implant selection, the position of the femoral and tibial resection surfaces, the amount of remaining healthy bone, and the patient's anatomy.

While surgeons can learn much about the state of the operative joint through pre-operative imagery (e.g., radiographs, computed tomography (“CT”) scans, magnetic resonance imaging (“MRI”)), certain information, especially information about radiolucent portions of the preoperative imagery (such as tendons, ligaments, and cartilage) and radiolucent portions of prior installed endoprosthetic implants (such as the tibial insert) is frequently only learned intraoperatively.

For example, a surgeon who is revising a knee (i.e., a surgeon who is replacing a prior installed “primary” endoprosthetic implant with a new endoprosthetic implant) may learn that the lateral side of the primary tibial insert wore at a faster rate than the medial side. This may be more likely to occur when the prior-implanted knee was placed using a surgical technique that sought to approximate or replicate the natural pre-diseased joint line of the patient (e.g., an anatomic alignment or a kinematic alignment surgical technique). To address this, the surgeon could alter the joint alignment (e.g., change the knee from a valgus knee to a mechanical knee or slight varus knee) to try to distribute the wear forces evenly. However, this approach would alter the patient's proprioception. That is, altering the joint alignment would alter the patient's joint position sense and the kinesthetic movement sense. As a result, the patient may have to learn how to walk with the new, altered joint line, which could lead to patient dissatisfaction and may prolong recovery.

Altering the joint alignment through successive bone recuts also generally adds time to the procedure. To recut bone, surgeons typically reassemble and re-align recut guides, possibly select differently sized trial implants, re-install the trial implants, and retest the patient's knee with the trial implants for a balanced flexion and extension range of motion. “Balance” can refer to the medial collateral ligament (“MCL”) and the lateral collateral ligament (“LCL”) being in equal tension throughout the range of flexion and extension motion of the knee, or the restoration of the native ligament tension depending on the implant design or the surgeon's alignment philosophy. Prolonged time under anesthesia can increase the risk of infection, surgical complications, and patient recovery time. Excessive bone recuts can also introduce avoidable debris into the surgical cavity, reduce the amount of remaining healthy bone, and further reduce error margins.

For these reasons, some surgeons instead try to reconstruct the patient's native joint line with the understanding that the uneven force distribution of the patient' native joint line may lead to accelerated wear of the tibial insert, which could lead to further revisions and additional surgery. Because the tibial insert is typically radiolucent, a revising surgeon may not appreciate the existence of or the extent of such wear until the surgeon is able to assess the joint intraoperatively.

Once the extent of wear is ascertained, the surgeon is generally limited by selecting standardized, unitary tibial inserts for the knee implant, which can again lead to the problems described above.

### SUMMARY OF THE INVENTION

The problems of the prior art, including the problem of uneven wear of the tibial insert, the problems associated with altering the patient's native joint line, and the problems associated with successive bone recuts, can be solved by an exemplary tibial insert comprising a medial component and a lateral component that further comprise specifications that can be independently assessed and selected based on the anatomy of a patient or a state of a prior implanted endoprosthetic implant.

By way of example, specifications for the medial component and the lateral component of an exemplary tibial insert can comprise an anterior-posterior (“AP”) slope angle, a medial-lateral (“ML”) slope angle, a minimum thickness, a size, a dwell point location, a geometry of an articular surface, a radius of curvature, an engagement mechanism, and a biocompatible material.

In one exemplary embodiment, a modular tibial insert assembly is provided. This modular tibial insert assembly comprises: a modular medial insert component, the modular medial insert component having a medial component proximal side proximally disposed from a medial component distal base side, wherein a medial component body extends between the medial component proximal side and the medial component distal base side, and a first set of medial component specifications; a modular lateral insert component, the modular lateral insert component having a lateral component proximal side proximally disposed from a lateral component distal base side, wherein a lateral component body extends between the lateral component proximal side and the lateral component distal base side, and a first set of lateral component specifications; wherein the modular tibial insert assembly has an assembled configuration and a disassembled configuration, and wherein the modular medial insert component is configured to fixedly engage the modular lateral insert component or a tibial component in the assembled configuration.

It is contemplated that in certain exemplary embodiments, the modular medial insert component and the modular lateral insert component can together comprise a “medial pivot” knee, which is believed to replicate the natural pivoting movement of the knee around the medial femoral and tibial condyles as the knee bends from extension to flexion more accurately than “J-curve” or “medial stabilized” knees.

It is contemplated that certain exemplary embodiments in accordance with the present disclosure may permit surgeons to implant an exemplary modular tibial insert assembly using any clinically recognized surgical method while constructing a tibial insert construct from a supply of modular medial insert components and a supply of modular lateral insert components having different desirable specifications that permit the functional reconstruction of a patient's pre-diseased knee through independently assessing and addressing variances or uneven force distribution in the medial side of the knee joint and the lateral side of the knee joint.

It is further contemplated that in certain circumstances, a surgeon replace a prior installed tibial insert in a revision procedure with an exemplary modular tibial insert assembly in accordance with this disclosure without removing a prior implanted tibial component, thereby avoiding the need for further sacrificing the proximal tibia.

It is further contemplated that in revising a prior implanted exemplary modular tibial insert assembly, a surgeon may elect to only remove a worn modular lateral insert component without having to replace the modular medial insert component or vice versa depending upon wear conditions.

### BRIEF DESCRIPTION OF THE DRAWINGS

The foregoing will be apparent from the following more particular description of exemplary embodiments of the disclosure, as illustrated in the accompanying drawings. The drawings are not necessarily to scale, with emphasis instead being placed upon illustrating the disclosed embodiments.

FIG. 1 is an isometric partially exploded view of an exemplary modular tibial insert assembly comprising a medial insert component, a lateral insert component, and a tibial component in a disassembled configuration.

FIG. 2 is a detailed view of the medial insert component of FIG. 1.

FIG. 3 is a detailed view of the lateral insert component of FIG. 1.

FIG. 4A is an isometric partially exploded view of several available modular insert components in a disassembled configuration, wherein the modular insert components have different slope angles and articular surfaces.

FIG. 4B is an isometric view of an exemplary modular tibial insert assembly shown in the assembled configuration together with a tibial component.

FIG. 5 is an isometric partially exploded view of an exemplary modular tibial insert assembly comprising a medial insert component, a lateral insert component, a tibial component, and a femoral component in a disassembled configuration.

FIG. 6A is an isometric view of an exemplary modular tibial insert assembly shown in the disassembled configuration, where the locking mechanism is a dovetail locking mechanism.

FIG. 6B is an isometric view of an exemplary modular tibial insert assembly shown in the disassembled configuration, wherein the locking mechanism comprises multiple dovetail locking mechanisms.

FIG. 7A is a top down view of an exemplary lateral insert component having an anterior-posterior slope angle.

FIG. 7B is a cross-sectional side view of the exemplary lateral insert component of FIG. 7A taken along the line A-A.

FIG. 8A is a top down view of an exemplary lateral insert component having a medial-lateral slope angle.

FIG. 8B is a cross-sectional side view of the exemplary lateral insert component of FIG. 8A taken along the line B-B.

FIG. 9A is a top down view of an exemplary medial insert component having a dwell point.

FIG. 9B is a cross-sectional side view of the exemplary medial insert component of FIG. 9A taken along the line C-C.

FIG. 10 is an anterior perspective view of an exemplary medial insert component and an exemplary lateral insert component comprising a dovetail locking mechanism, wherein the medial component comprises a flat surface, which allows the medial femoral condyle to slide and rotate posteriorly during high knee flexion.

FIG. 11A is an anterior and partially superior facing view of an exemplary modular tibial insert assembly comprising a medial insert component, a lateral insert component, and a tibial component in a disassembled configuration, wherein the medial insert component and the lateral insert component are configured to engage the tibial component without also mechanically engaging each other.

FIG. 11B is an anterior and partially inferior facing view of the exemplary modular tibial insert assembly of FIG. 11A shown in a partially assembled configuration.

FIG. 12A is an anterior exploded view of an exemplary CCK type modular tibial insert assembly shown in a disassembled configuration.

FIG. 12B is an anterior and partially inferior facing perspective view of the exemplary CCK modular tibial insert assembly of FIG. 12A shown in an assembled configuration.

### BRIEF DESCRIPTION OF THE PREFERRED EMBODIMENTS

The following detailed description of the preferred embodiments is presented only for illustrative and descriptive purposes and is not intended to be exhaustive or to limit the scope and spirit of the invention. The embodiments were selected and described to best explain the principles of the invention and its practical application. One of ordinary skill in the art will recognize that many variations can be made to the invention disclosed in this specification without departing from the scope and spirit of the invention.

Similar or same reference characters indicate corresponding parts throughout the several views unless otherwise stated. Although the drawings represent embodiments of various features and components according to the present disclosure, the drawings are not necessarily to scale and certain features may be exaggerated to better illustrate embodiments of the present disclosure, and such exemplifications are not to be construed as limiting the scope of the present disclosure.

Except as otherwise expressly stated herein, the following rules of interpretation apply to this specification: (a) all words used herein shall be construed to be of such gender or number (singular or plural) as such circumstances require; (b) the singular terms “a,” “an,” and “the,” as used in the specification and the appended claims include plural references unless the context clearly dictates otherwise; (c) the antecedent term “about” applied to a recited range or value denotes an approximation with the deviation in the range or values known or expected in the art from the measurements; (d) the words, “herein,” “hereby,” “hereto,” “hereinbefore,” and “hereinafter,” and words of similar import, refer to this specification in its entirety and not to any particular paragraph, claim, or other subdivision, unless otherwise specified; (e) descriptive headings are for convenience only and shall not control or affect the meaning of construction of part of the specification; and (f) “or” and “any” are not exclusive and “include” and “including” are not limiting. Further, the terms, “comprising,” “having,” “including,” and “containing” are to be construed as open-ended terms (i.e., meaning “including but not limited to”).

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments, whether explicitly described.

To the extent necessary to provide descriptive support, the subject matter and/or text of the appended claims are incorporated herein by reference in their entirety.

Recitation of ranges of values herein are merely intended to serve as a shorthand method of referring individually to each separate value falling within the range of any sub-ranges there between, unless otherwise clearly indicated herein. Each separate value within a recited range is incorporated into the specification or claims as if each separate value were individually recited herein. Where a specific range of values is provided, it is understood that each intervening value, to the tenth or less of the unit of the lower limit between the upper and lower limit of that range and any other stated or intervening value in that stated range of sub range thereof, is included herein unless the context clearly dictates otherwise. All subranges are also included. The upper and lower limits of these smaller ranges are also included therein, subject to any specifically and expressly excluded limit in the stated range.

The terms, “horizontal” and “vertical” are used to indicate direction relative to an absolute reference, i.e., ground level. However, these terms should not be construed to require structure to be absolutely parallel or absolutely perpendicular to each other. For example, a first vertical structure and a second vertical structure are not necessarily parallel to each other.

Throughout this disclosure and unless otherwise noted, various positional terms, such as “distal,” “proximal,” “medial,” “lateral,” “anterior,” and “posterior,” will be used in the customary manner when referring to the human anatomy. More specifically, “distal” refers to the area away from the point of attachment to the body, while “proximal” refers to the area near the point of attachment to the body. For example, the distal femur refers to the portion of the femur near the tibia, whereas the proximal femur refers to the portion of the femur near the hip. The terms, “medial” and “lateral” are also essentially opposites. “Medial” refers to something that is disposed closer to the middle of the body. “Lateral” means that something is disposed closer to the right side or the left side of the body than to the middle of the body. Regarding, “anterior” and “posterior,” “anterior” refers to something disposed closer to the front of the body, whereas “posterior” refers to something disposed closer to the rear of the body.”

“Varus” and “valgus” are broad terms and include without limitation, rotational movement in a medial and/or lateral direction relative to the knee joint.

The term, “mechanical axis” of the femur refers to an imaginary line drawn from the center of the femoral head to the center of the distal femur at the knee.

The term, “anatomic axis” refers to an imaginary line drawn lengthwise down the middle of femoral shaft or tibial shaft, depending upon use.

Briefly, in a primary total knee arthroplasty (“TKA”), the surgeon typically makes a vertical medial parapatellar incision of about six to about ten inches (i.e., about 15.24 centimeters to about 25.4 centimeters) in length on the anterior or anteromedial aspect of the knee. The surgeon then continues to incise the fatty tissue to expose the anterior or anteromedial aspect of the joint capsule. The surgeon may then perform a medial parapatellar arthrotomy to pierce the joint capsule. A retractor may then be used to move the patella generally laterally (roughly about 90 degrees) to expose the distal condyles of the femur and the cartilaginous meniscus resting on the proximal tibial plateau. The surgeon then removes the meniscus and uses instrumentation to measure and resect the distal femur and proximal tibia to accommodate trial implants.

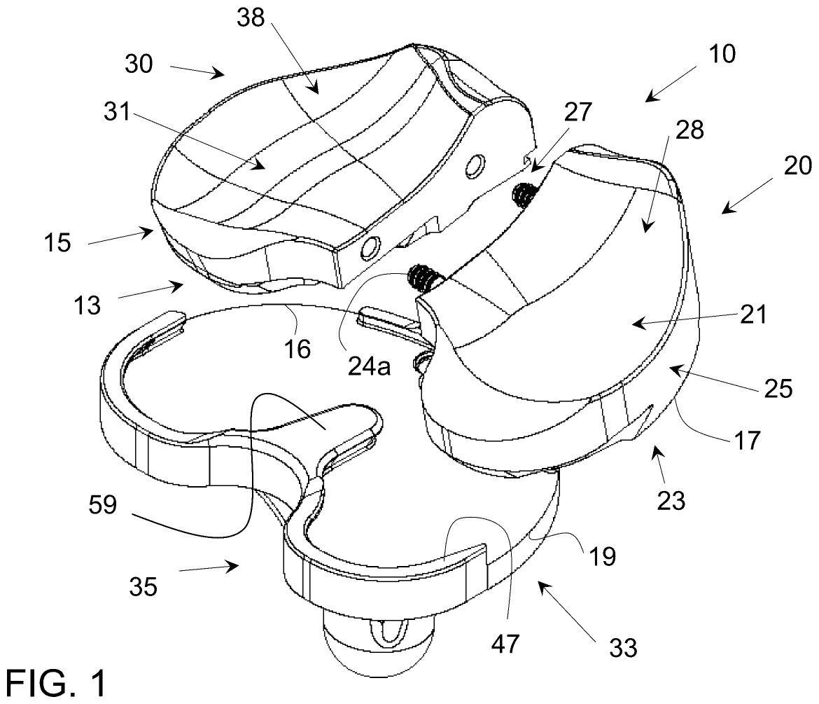

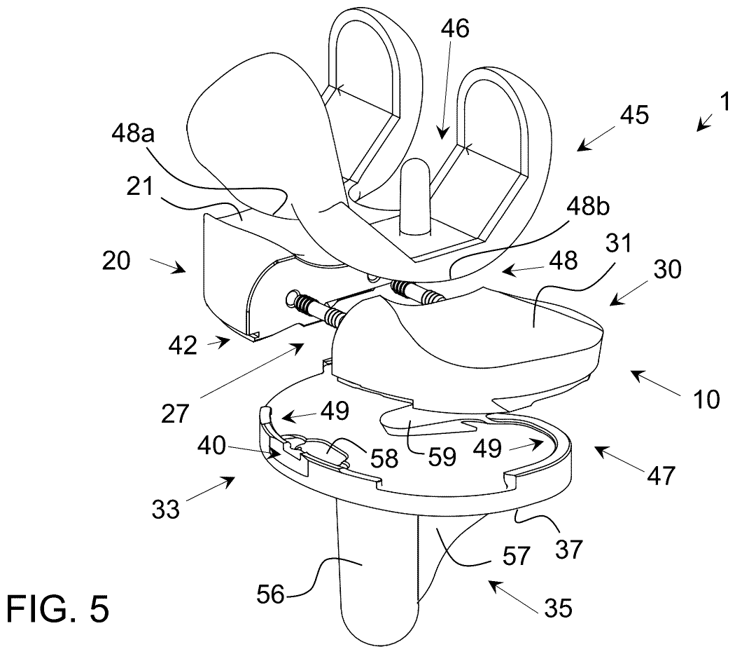

The resections themselves often remove areas of diseased bone. These resections also necessarily modify the profiles of the remaining distal femur and proximal tibia to better accommodate complementary shapes of the associated implant component. That is, the resected distal femur will eventually abut against the inner surfaces that form the concave portion 46 (FIG. 5) of a complementary femoral component 45 (FIG. 5). Likewise, the resected proximal tibia will eventually support the distal surface 37 (FIG. 5) of a complementary tibial component 35 (FIG. 1).

Ultimately, a final endoprosthetic implant will be selected and assembled based on the sizing and the movement mechanics of the trial implants. The final implant typically comprises a femoral component 45 that is placed on, and sometimes partially in, the resected distal femur, a tibial component 35 that is placed on and partially in the resected proximal tibia, and an insert (typically known as a “tibial insert,” “a poly,” or a “meniscal insert”) that is disposed between the implanted femoral component 45 and the implanted tibial component 35. The insert generally has a superior articular surface (see 21, 31, FIG. 1) that functions as a bearing surface against which the distal femoral condyles 48 (FIG. 5) of the femoral component 45 rotate to flex and extend the knee. The distal femoral condyles 48 comprise a medial distal condyle 48a medially disposed from a lateral distal condyle 48b.

A surgeon typically selects from one of several types of tibial insert. A cruciate substituting (“CS”) tibial insert is typically used when the surgeon resects both the anterior cruciate ligament (“ACL”) and the posterior cruciate ligament (“PCL”). A cruciate retaining (“CR”) tibial insert is typically selected when the surgeon resects only the ACL but preserves the PCL, and a posterior stabilized (“PS”) tibial insert is typically selected when the surgeon resects all cruciate ligaments, including the ACL, PCL, MCL, and LCL. PS tibial inserts are characterized by a slightly rounded post extending from the articular surface, which generally serves to replace the function of the MCL and LCL. A constrained condylar knee (“CCK”) tibial insert is similar to a PS insert except that the post is generally more rectangular with flat medial and lateral post surfaces configured to reduce internal and external rotation of the knee around a generally vertical axis.

Several schools of thought have arisen concerning how and where to resect the distal femoral condyles and the proximal tibia.

One school of thought is the anatomic alignment method. In the anatomic alignment method, the surgeon resects the tibia at 3 degrees of varus because this is believed to be the average angle of the native joint line. Femoral resections and ligament releases are then performed to keep a straight hip-knee-ankle axis of the limb.

Another school of thought is the mechanical alignment method. The goal of mechanical alignment is to resect the tibia perpendicular to the axis of the tibial shaft (i.e., parallel to the transverse body plane). A mechanically resected tibial plateau would have a 0 degree varus tilt. The distal femur is then adjusted to account for the 0 degree varus tilt of the resected tibia and any necessary ligament releases are performed to maintain a straight hip-knee-ankle axis.

By contrast, with the kinematic alignment method, the surgeon seeks to restore the patient's specific natural pre-diseased joint line based on data made available to the surgeon pre-operatively, intra-operatively, or a combination thereof. Many kinematic alignment techniques start with referencing the distal femur and generally adjusting the slope of the tibial resection(s) to be parallel to the distal femoral resection when the knee is in extension, and parallel to the posterior resections of the femoral condyles when the knee is in flexion.

The placement of these final implant components, and by extension, the location of the resected surfaces upon which the respective implant components are disposed and engaged, largely dictates the position of the reconstructed joint line. The angles of resection and the precise placement of the implant components on the resected surfaces can also influence how the artificial joint performs over time. For example, using the kinematic alignment philosophy, a surgeon may use instrumentation to ascertain that a patient's native pre-diseased joint line was disposed at four degrees varus and elect to resect the distal femur and proximal tibia at four degrees varus to replicate the pre-diseased joint line. In this example, the reconstructed pre-diseased joint line should feel natural to the patient and mitigate the pain and discomfort that can accompany relearning how to walk with altered anatomy. However, at a varus angle, more force above the distal femur may be transferred to the proximal tibial and ultimately the feet via the medial femoral and tibial condyles than the lateral femoral and distal condyles, which can result in uneven wear of the medial side of the tibial insert compared to the lateral side of the tibial insert. It will be appreciated that in reconstructed valgus knees, the lateral side of the tibial insert may wear at a greater rate than the medial side of the tibial insert.

A revision TKA is similar to a primary TKA except that after the surgeon has exposed the knee joint, the surgeon removes the prior-installed primary endoprosthetic implant. To do this, the surgeon generally resects additional bone below the primary tibial component and above the primary femoral component 45 and then uses revision endoprosthetic knee implants to compensate for the additional bone loss.

FIG. 1 is an isometric partially exploded view of an exemplary cruciate substituting (“CS”) modular tibial insert assembly 10 depicted in a disassembled configuration. The exemplary modular tibial insert assembly 10 comprises a modular medial tibial insert component 20 (referred to periodically throughout as a “modular medial insert component 20” or a “medial insert component 20” unless otherwise specified), a modular lateral tibial insert component 30 (referred to periodically throughout as a “modular lateral insert component 30” or a “lateral insert component 30” unless otherwise specified). In certain exemplary embodiments, the modular tibial insert assembly 10 further comprises a tibial base component 35.

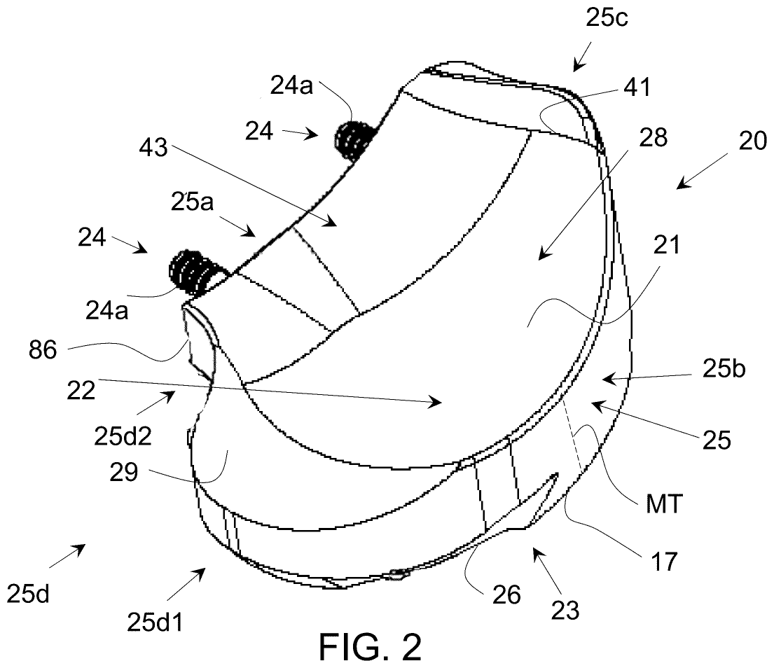

Relational elements to the modular medial insert component 20 are provided with reference to the modular medial insert component 20 as if the modular medial insert component 20 were oriented in an orientation consistent with the modular medial insert component's orientation in the assembled and implanted configuration. The modular medial insert component 20 has a proximal side 28 (e.g., a medial insert component first side) proximally disposed from a medial component distal base side 23 (e.g., a medial insert component second side). A medial component body 25 extends between the medial component proximal side 28 and the medial component distal base side 23. Referring to FIG. 2, the body 25 is further bounded by a medial component inner periphery side 25a, a medial component outer periphery side 25b, the medial component outer periphery side 25b being medially disposed from the medial component inner periphery side 25a, a medial component anterior periphery side 25c anteriorly disposed between the medial component inner periphery side 25a and the medial component outer periphery side 15b, and a medial component posterior periphery side 25d posteriorly disposed from the medial component anterior periphery side 25c.

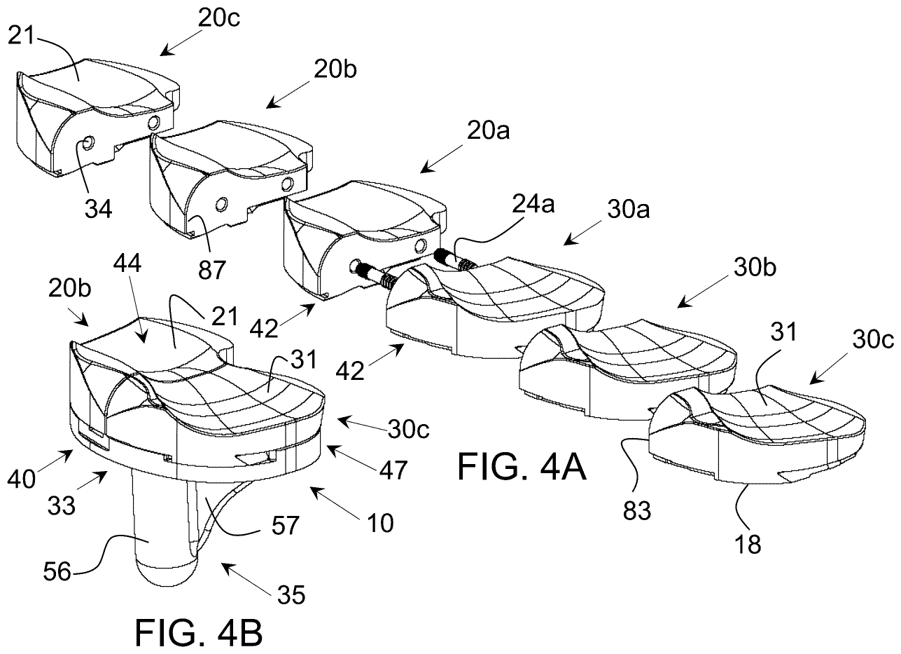

In the depicted embodiment, a posterior edge 86 separates the medial component posterior periphery side 25d from the medial component inner periphery side 25a. An anterior edge 87 (FIG. 4A) separates the medial component anterior periphery side 25c from the medial component inner periphery side 25a.

The medial component proximal side 28 comprises a medial component articular surface 21. When the endoprosthetic knee implant 1 (FIG. 5) is fully assembled and implanted, the distal medial femoral condyle 48a of the femoral component 45 rotates against this medial component articular surface 21 as the knee implant 1 bends from extension to flexion. The medial component distal base side 23 comprises a medial component distal base surface 26 (FIG. 2). The medial component body 25 has a thickness MT (FIG. 2). In certain exemplary embodiments, the thickness MT of the medial component body 25 is measured from the lowest point on the medial component proximal side 28 to the highest point of surface on the medial component distal base side 23 (see FIG. 2). In other exemplary embodiments, the thickness MT of the medial component body 25 is measured from the highest point of the medial component proximal side 28 to the lowest point on the medial component distal base side 23. In still other exemplary embodiments, the thickness MT of the medial component body 25 is an average of the thicknesses of the body 25 of the modular medial insert component 20. In still other exemplary embodiments, the thickness MT of the medial component body 25 is measured from a geometric midpoint of the modular medial insert component 20. In still other exemplary embodiments, a minimum thickness is measured from the dwell point to the nearest portion of the medial component distal base side 23.

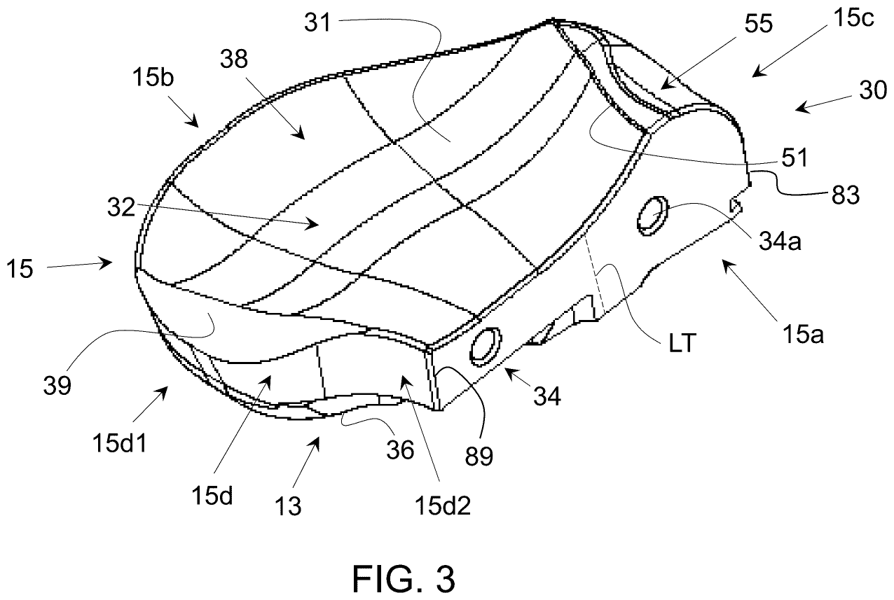

Relational elements to the modular lateral insert component 30 are provided with reference to the modular lateral insert component 30 as if the modular lateral insert component 30 were oriented in an orientation consistent with the modular lateral insert component's orientation in the assembled and implanted configuration. The modular lateral insert component 30 has a proximal side 38 (e.g., a lateral insert component first side) proximally disposed from a lateral component distal base side 13 (e.g., a lateral insert component second side). A lateral component body 15 extends between the lateral component proximal side 38 and the lateral component distal base side 13. Referring to FIG. 3, the body 15 is further bounded by a lateral component inner periphery side 15a, a lateral component outer periphery side 15b, the lateral component outer periphery side 15b being laterally disposed from the lateral component inner periphery side 15a, a lateral component anterior periphery side 15c anteriorly disposed between the lateral component inner periphery side 15a and the lateral component outer periphery side 15b, and a lateral component posterior periphery side 15d posteriorly disposed from the lateral component anterior periphery side 15c.

In the depicted embodiment, a posterior edge 89 separates the lateral component posterior periphery side 15d from the lateral component inner periphery side 15a. An anterior edge 83 (FIG. 4A) separates the lateral component anterior periphery side 15c from the lateral component inner periphery side 15a.

The lateral component proximal side 38 comprises a lateral component articular surface 31. When the endoprosthetic knee implant 1 (FIG. 5) is fully assembled and implanted, the distal lateral femoral condyle 48b of the femoral component 45 rotates against this lateral component articular surface 31 as the knee implant 1 bends from extension to flexion. The lateral component distal base side 13 comprises a lateral component distal base surface 36. The lateral component body 15 has a thickness LT (FIG. 3). In certain exemplary embodiments, the thickness LT of the lateral component body 15 is measured from the lowest point on the lateral component proximal side 38 to the highest point of surface on the lateral component distal base side 13. In other exemplary embodiments, the thickness LT of the lateral component body 15 is measured from the highest point of the lateral component proximal side 38 to the lowest point on the lateral component distal base side 13. In still other exemplary embodiments, the thickness LT of the lateral component body 15 is an average of the thicknesses of the body 15 of the modular lateral insert component 30. In still other exemplary embodiments, the thickness LT of the lateral component body 15 is measured from a geometric midpoint of the modular lateral insert component 30. In still other exemplary embodiments, a minimum thickness is measured from the lowest portion of the lateral component articular surface 31 to the nearest portion of the lateral portion distal base side 23.

As better seen in the detailed view of FIG. 2, the body 25 of the modular medial insert component 20 comprises a medial component inner periphery side 25a and a medial component outer periphery side 25b medially disposed from the medial component inner periphery side 25a when oriented in the assembled and implanted configuration. Likewise, the detailed view of FIG. 3 shows that the lateral component body 15 of the modular lateral insert component 30 comprises a lateral component inner periphery side 15a and a lateral component outer periphery side 15b laterally disposed from the lateral component inner periphery side 15a when oriented in the assembled and implanted configuration.

Referring to FIGS. 1-3, the locking mechanism 27 that is configured to fixedly engage the modular medial insert component 20 to the modular lateral insert component 30 (FIG. 1) is a projection-receiver engagement mechanism. In the depicted embodiment, the projections 24 comprise locking fasteners 24a. These locking fasteners 24a can be removable projections (see FIG. 4) in some embodiments. In other embodiments, the projection 24 can be integral to either the modular medial insert component 20 or the modular lateral insert component 30. As shown in the exemplary embodiment of FIG. 2, the projection 24 extends from medial component inner periphery side 25a and is received by the receivers 34 (e.g., holes 34a) disposed in the lateral component and communicating with the lateral component inner periphery side 15a (FIG. 3) in the assembled configuration.

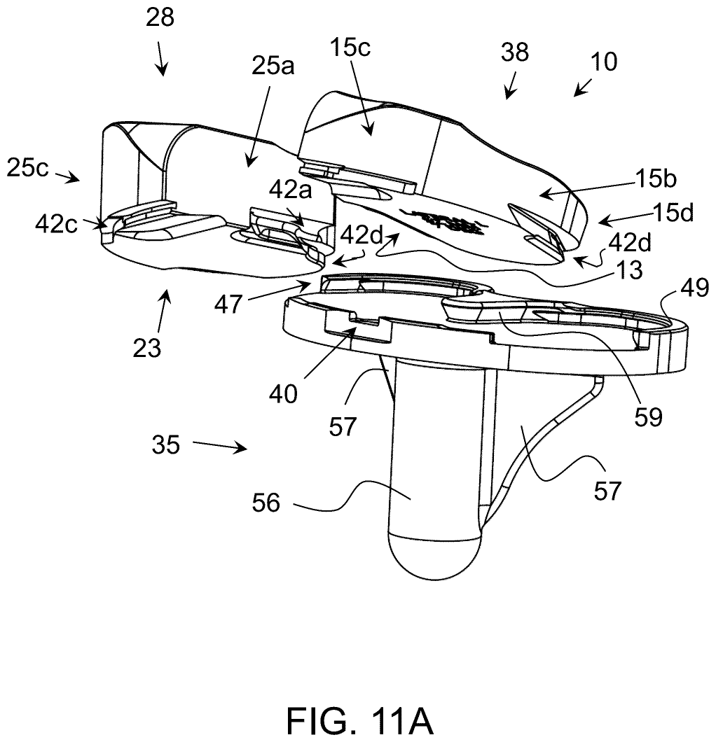

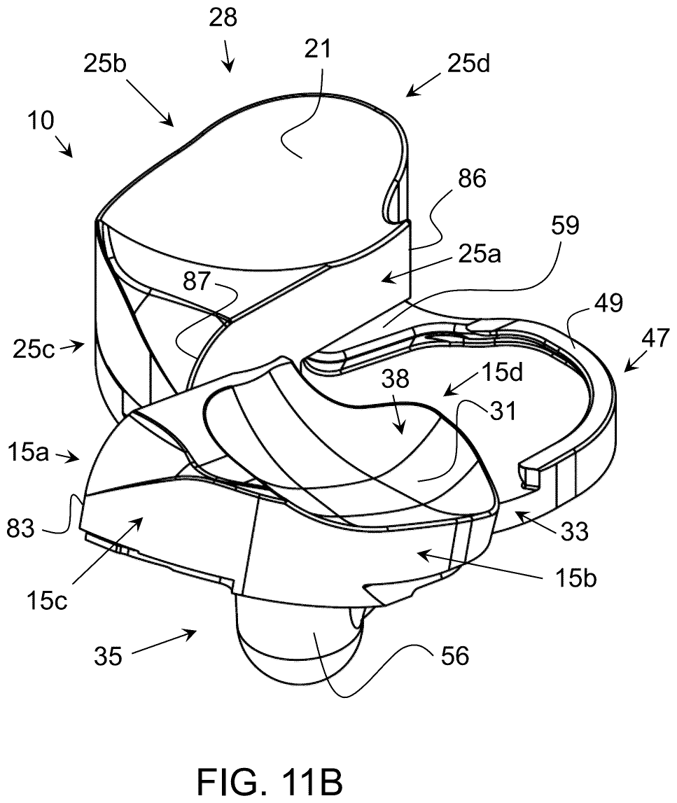

Although a projection-receiver locking mechanism 27 is shown, nothing in this disclosure limits the locking mechanism 27 to be a projection-receiver locking mechanism. Other examples of locking mechanisms 27 include, but are not limited to, magnets of opposite polarity, an adhesive, a bonding agent, a thermal engagement mechanism, or combinations or permutations the above described locking mechanisms. Furthermore, the locking mechanism 27 need not be disposed between the medial component inner periphery side 25a and the lateral component inner periphery side 15a in every embodiment. In other exemplary embodiments, the medial insert component 20 or the lateral insert component 30, or both the medial insert component 20 and the lateral insert component 30 can have locking mechanisms 27 configured to fixedly engage the medial insert component 20, the lateral insert component 30, or both the medial insert component 20 and the lateral insert component 30 to the tibial base component 35 (see FIGS. 11A and 11B).

It will be appreciated that a modular medial insert component 20 can be said to be “configured to fixedly engage” the modular lateral insert component 30 (and vice versa) in the assembled configuration (see FIG. 4) when a locking mechanism 27 is present that, when the locking mechanism 27 is engaged, substantially prevents translational movement of the modular medial insert component 20 relative to the modular lateral insert component 30. It will be appreciated that a locking mechanism 27 can permit the direct engagement of the modular medial insert component 20 to the modular lateral insert component 30 in certain exemplary embodiments. In other exemplary embodiments, the locking mechanism 27 can permit the indirect engagement of the modular medial insert component 20 to the modular lateral insert component 30 through an intermediary construct. In still other exemplary embodiments, a retaining wall 47 of tibial base component 35 can serve as a locking mechanism 27 for the modular medial insert component 20 and the modular lateral insert component 30 (see FIGS. 11A and 11B). Combinations of the foregoing are considered to be within the scope of this disclosure.

In embodiments wherein the modular insert components 20, 30 are “configured to fixedly engage” each other (e.g., either directly or indirectly), the “assembled configuration” means the position that the modular medial insert component 20 and the modular lateral insert component 30 are in when the modular medial insert component 20 and the modular lateral insert component 30 fixedly engage each other. Likewise, a “disassembled configuration” means the positions that the modular medial insert component 20 and the modular lateral insert component 30 are in when they are not fixedly engaged to each other. Furthermore an “implanted configuration” means the position that the modular medial insert component 20 and the modular lateral insert component 30 are disposed in when assembled on the tibial component 35 and surgically implanted into a patient's tibia.

Although a modular medial insert component 20 that is configured to be fixedly engaged to a modular lateral insert component 30 (and vice versa) is depicted in FIGS. 1-5, it will be appreciated that in other exemplary embodiments, the modular insert components 20, 30 can be configured to fixedly engage the tibial base component 35. In certain exemplary embodiments, the modular insert components 20, 30 can fixedly engage the tibial base component 35 instead of or in addition to fixedly engaging each other in an assembled configuration (see FIGS. 11A and 11B). In such embodiments, the “assembled configuration” means the position that the modular medial insert component 20 and the modular lateral insert component 30 are in when the modular medial insert component 20 and the modular lateral insert component 30 fixedly engage (e.g., either directly or indirectly) the tibial base component 35. In other exemplary embodiments, the locking mechanism 27 can permit the indirect engagement of the modular medial insert component 20, modular lateral insert component 30, or both the modular medial insert component 20 and modular lateral insert component 30 to the tibial baseplate 33 of the tibial component 35 through an intermediary construct. Likewise, a “disassembled configuration” means the positions that the modular medial insert component 20 and the modular lateral insert component 30 are in when they are not fixedly engaged to the tibial base component 35. In other exemplary embodiments, one or more of the modular insert components 20, 30 can be configured to fixedly engage the tibial base component 35 in addition to being configured to fixedly engage each other. Combinations and permutations of the foregoing are considered to be within the scope of this disclosure.

Referring to FIG. 2, the depicted medial component articular surface 21 of the exemplary modular medial insert component 20 has a curved concave portion 22 that contacts a mated medial condyle of the femoral component 45 when the medial pivot endoprosthetic implant 1 (FIG. 5) is fully assembled and implanted. The curved concave portion 22 is disposed closer to the outer periphery side 25b than the inner periphery side 25a. The medial component proximal side 28 further comprises a lateral constraint portion 43 disposed between the medial component inner periphery side 25a and the articular surface 21. In certain exemplary embodiments, the curved concave portion 22 of the articular surface 21 is substantially spherical (i.e., has a substantially constant radius of curvature). Without being bound by theory, it is contemplated that a substantially spherical curved concave portion 22 disposed against a mated medial condyle of the femoral component 45 provides stability to the knee and permits the endoprosthetic knee implant to pivot via the modular medial insert component 20 and the medial femoral condyle as the knee bends from extension to flexion. In this manner, these components of a fully assembled and implanted endoprosthetic implant are thought to enable the replication of the pivoting kinematic movement of the natural knee. A modular medial insert component 20 that comprises an articular surface 21 that permits the medial femoral condyle to pivot around a substantially vertical axis in the sagittal plane in an assembled and surgically implanted position can be said to be a “medial pivot medial component articular surface.” A “medial pivot component articular surface” further desirably comprise a portion with a constant radius of curvature to facilitate the pivoting and flexion-extension motion.

The exemplary modular medial insert components 20 of FIGS. 2 and 3 are a CS modular medial insert component 20 and a CS modular lateral insert component 30, respectively. However, it will be appreciated that modular medial inserts and modular lateral inserts configured for CR tibial inserts, PS tibial inserts, and CCK tibial inserts are considered to be within the scope of this disclosure.

Referring to FIG. 3, the depicted lateral component articular surface 31 of the modular lateral insert component 30 comprises a partial toroidal concave portion 32 that contacts the mated lateral condyle of the femoral component 45 when the medial pivot endoprosthetic implant is fully assembled and implanted. In certain exemplary embodiments, the partial toroidal concave portion 32 permits about 15 degrees of arcuate lateral femoral condyle motion as the knee moves from extension to flexion relative to the lateral component articular surface 31 and relative to the substantially spherical curved concave portion 22 of the modular medial insert component 20 when an exemplary medial pivot knee endoprosthetic implant is fully assembled. It will be appreciated that other exemplary lateral component articular surfaces 31 may comprise a partial toroidal concave portion 32 that permits between about 10 degrees of arcuate lateral femoral condyle motion to about 25 degrees of arcuate lateral femoral motion. A medial pivot angle can be envisioned by imaging the angle formed by an imaginary line extending from the rotational (not flexional) point of contact between the medial femoral condyle 48a and the medial component articular surface 21 and the point of contact between the lateral femoral condyle 48b and the lateral component articular surface 31 when the knee is in extension and the imaginary line formed by the same contact points when the knee is in flexion.

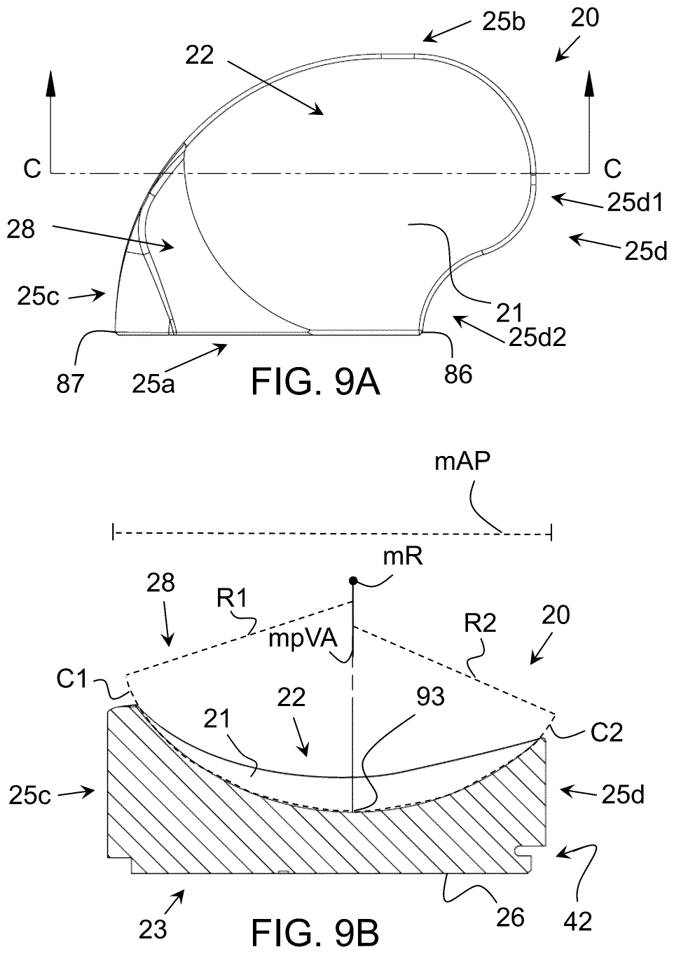

Without being bound by theory, it is contemplated that lateral component articular surface 31 having a partial toroidal concave portion 32, when used in conjunction with a modular medial insert component 20 in the assembled configuration as described herein, permits the medial femoral condyle of the endoprosthetic knee implant to pivot around a substantially medial pivot vertical axis mpVA extending from the dwell point 93 (FIG. 9B) via the modular medial insert component 20. This pivoting movement (i.e., internal-external rotation) the medial femoral condyle 48a relative to the medial tibial insert component 20 causes the lateral femoral condyle 48b to move in a generally arcuate path along the lateral component articular surface 31 as the femoral component 45 of an exemplary endoprosthetic knee implant bends from extension to flexion relative to an exemplary modular tibial insert assembly 10. That is, the contact point between the distal lateral femoral condyle 48b and the lateral component articular surface 31 can be visualized as moving in a generally arcuate path along the partial toroidal concave portion 32 of the lateral component articular surface 31 as the knee implant 1 bends from extension to flexion. In this manner, these components of a fully assembled and implanted endoprosthetic implant are thought to enable the replication of the pivoting kinematic movement of the natural knee. Stated differently, the natural knee is believed not to be a simple hinge, but rather a complex bending joint that comprises both a transverse bending axis (i.e., a flexion-extension axis) and a medial pivoting axis (mpVA) that permits internal-external rotation around this medial pivoting axis as the knee bends around the flexion-extension axis. A modular lateral insert component 30 that comprises a lateral component articular surface 31 that permits the lateral femoral condyle to move in an arcuate path around the modular medial insert component 20 when positioned in an assembled configuration and surgically implanted position can be said to be a “medial pivot lateral component articular surface.”

It will be appreciated that nothing in this disclosure limits the modular medial and lateral insert components 20, 30 to be medial pivot medial and lateral components 20, 30. In other exemplary embodiments, the modular medial and lateral components 20, 30 are symmetric and have symmetric articular surfaces 21, 31. In yet other exemplary embodiments, the modular medial and lateral components 20, 30 have dual pivot articular surfaces 21, 31. In still yet other exemplary embodiments, the modular medial and lateral components 20, 30 have dual dish articular surfaces 21, 31. In still yet further exemplary embodiments, the modular medial and lateral components 20, 30 have asymmetric non-medial pivot articular surfaces 21, 31. In still yet further exemplary embodiments, the modular medial and lateral components 20, 30 have lateral pivot articular surfaces 21, 31. Combinations and permutations of the foregoing (e.g., a medial pivot medial component 20 and an asymmetric non-medial pivot lateral component 30) are considered to be within the scope of this disclosure.

Referring back to the embodiment depicted in FIG. 2, the medial component proximal side 28 desirably further comprises a medial component ACL posterior lip 29 located posteriorly to the curved concave portion 22 of the medial component articular surface 21. Without being bound by theory, it is contemplated that the medial component ACL posterior lip 29 prevents posterior slide of the medial femoral condyle in knee flexion and thereby serves a similar function as the native ACL, which is sacrificed to accommodate the implant. Likewise, a medial component anterior lip 41 located anterior to the curved concave portion 22 of the medial component articular surface 21 is thought to prevent anterior slide of the medial femoral condyle in extension. In other exemplary embodiments, the medial component articular surface 21 comprises a posterior portion with a convex area to permit posterior rollback in deep flexion. The medial component posterior periphery side 25d generally comprises an S-shaped profile having a convex portion 25d1 adjacently medially disposed from a concave portion 25d2. This S-shaped profile generally permits the medial component posterior periphery side 25d to conform to the profile of the resected tibia's medial posterior profile. It will be appreciated that in embodiments in which the modular medial insert component 20 is a CR modular medial insert component 20, the concave portion 25d2 may further be sloped with the base of the slope starting closer to the medial component distal base side 23 and extending in the anterior direction to accommodate the retained PCL.

Referring back to the embodiment depicted in FIG. 3, The lateral component proximal side 38 desirably further comprises a lateral component ACL posterior lip 39 located posteriorly to the partial toroidal concave portion 32 of the lateral component articular surface 31. Without being bound by theory, it is contemplated that the lateral component ACL posterior lip 39 prevents posterior slide of the lateral femoral condyle in knee flexion and thereby serves a similar function as the native ACL. Likewise, a lateral component anterior lip 51 located anterior to the partial toroidal concave portion 32 of the lateral component articular surface 31 is thought to prevent anterior slide of the lateral femoral condyle in extension to an extent. The lateral component anterior lip 51 is usually desirably lower than the medial component anterior lip 41 because the knee has been shown to pivot laterally slightly in full extension. The lower lateral component anterior lip 51 permits the femoral lateral condyle to traverse the lateral component anterior lip 51 as the knee moves to full extension before resting in the lateral anterior concave pocket 55 in full extension. The presence of the lateral femoral condyle in the lateral anterior concave pocket 55 in full extension permits the medial femoral condyle to pivot slightly around the lateral anterior concave pocket 55 in full extension, thereby replicating full kinematic movement of the natural knee.

In other exemplary embodiments, the lateral component articular surface 31 comprises a posterior portion with a convex area to permit posterior rollback in deep flexion.

As seen in the detailed view of FIG. 3, the body 15 of the modular lateral insert component 30 can comprise a lateral component inner periphery side 15a and a lateral component outer periphery side 15b laterally disposed from the lateral component inner periphery side 15a when oriented in the assembled configuration. In the depicted embodiment, a portion of a locking mechanism 27b extends into the lateral component inner periphery side 15a. In the depicted embodiment, the locking mechanism 27 is a projection-receiver engagement mechanism and the receivers 34 that extends into the lateral component inner periphery side 15a are holes 34a that are sized to closely receive the projections 24 depicted as extending from the medial component inner periphery side 15a of the modular medial insert component 20.

The lateral component posterior periphery side 15d generally comprises an S-shaped profile having a convex portion 15d1 adjacently medially disposed from a concave portion 15d2. This S-shaped profile generally permits the lateral component posterior periphery side 15d to conform to the profile of the resected tibia's lateral posterior profile. It will be appreciated that in embodiments in which the modular lateral insert component 30 is a CR modular lateral insert component 30, the concave portion 15d2 may further be sloped with the base of the slope starting closer to the lateral component distal base side 13 and extending in the anterior direction to accommodate the retained PCL.

Referring to FIGS. 1-6B, and 10 in the depicted embodiment, the medial component inner periphery side 25a physically abuts the lateral component inner periphery side 15a in the assembled configuration. A retaining wall 47 (FIGS. 4B, 5, 11A, 11B) of tibial base component 35 engages recesses (see posterior distal receiver 42d, anterior distal receiver 42c, and inner distal receiver 42a in FIG. 11A) in the distal base sides 13, 23 of the modular medial and lateral components 20, 30 in the assembled configuration, thereby fixedly engaging the modular medial insert component 20 and the modular lateral insert component 30 to the tibial baseplate 33. A medial distal edge 17 of the medial component outer periphery side 25b abuts a medial peripheral edge 19 (FIG. 1) of a tibial base component 35, and a lateral distal edge 18 (FIG. 4A) of the lateral component outer periphery side 15b abuts a lateral peripheral edge 16 (FIG. 1) of the tibial base component 35 in the assembled configuration. Aligning the lateral component outer periphery side 15b and the medial component outer periphery side 25b with the peripheral edges 16, 19 of the tibial baseplate 33 is thought to reduce the possibility for tissue impingement after surgery. Without being bound by theory, it is contemplated that assembling the modular medial and lateral inserts 20, 30 in any manner described herein provides the surgeon with options to recreate a joint line that most aligns with the patient's needs and the surgeon's professional judgment while reducing the potential for uneven wear due to uneven force distribution from the femoral condyles and the need for excessive bone recuts to adjust the joint alignment angle.

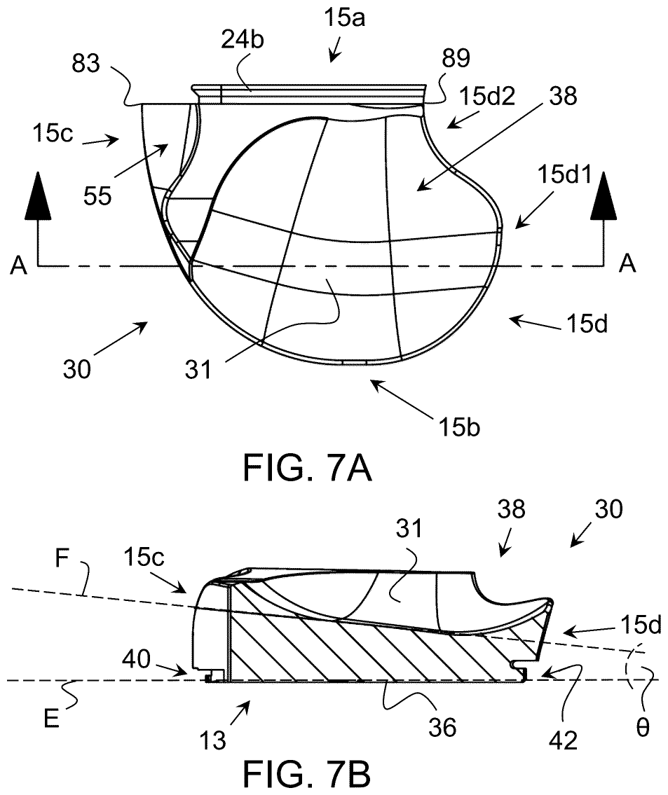

FIG. 4A is an isometric partially exploded view of several available modular medial insert components 20a, 20b, 20c and several available modular lateral insert components 30a, 30b, 30c in a disassembled configuration. The depicted modular medial insert components 20 and modular lateral insert components 30 have different anterior-posterior (“AP”) slope angles θ (FIG. 7B) relative to one another. In this manner, a surgeon can implant an exemplary endoprosthetic implant assembly using the alignment technique of the surgeon's choosing and further set the AP slope angles θ of the modular medial insert component 20 and the modular lateral insert component 30 independently to better accommodate the desired reconstructed knee kinematics of the patient.

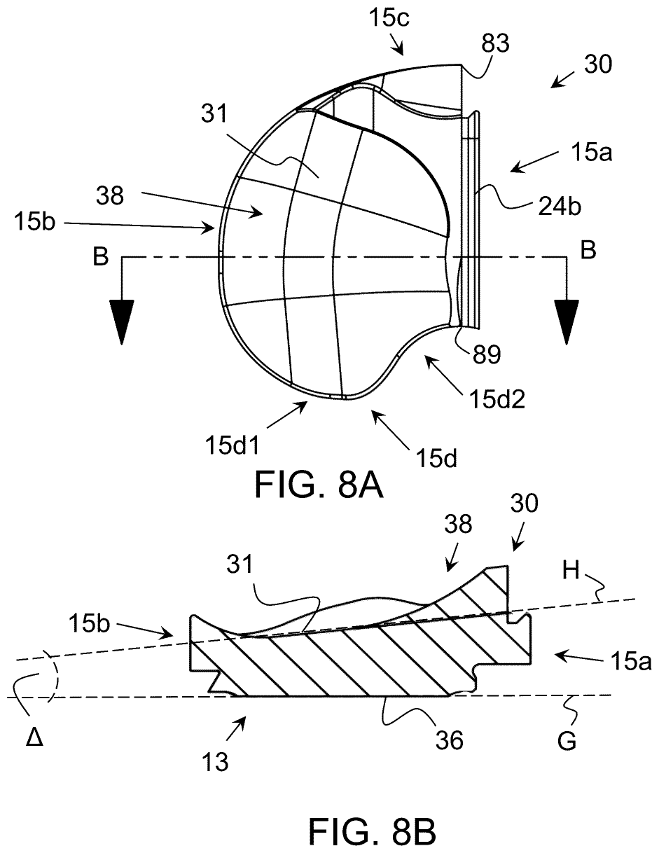

Although different AP slope angles θ are depicted, it will be appreciated that multiple modular insert components 20, 30 can be provided that have a number of different physical or material attributes (i.e., specifications). Such specifications can include different: geometries of articular surfaces 21, 31, sizes, thicknesses MT, LT, minimum thicknesses, medial-lateral (“ML”) slope angles Δ (see FIG. 8A and 8B), physical materials, dwell points, radii of curvatures, concavity, convexity, modular insert to modular insert locking mechanisms, modular insert to tibial baseplate engagement mechanisms, or combinations of any of the foregoing.

Without being bound by theory, it is contemplated that exemplary modular medial pivot tibial insert assemblies as described herein may permit surgeons or other medical professionals to position exemplary endoprosthetic implant assemblies and construct a tibial insert comprising a modular medial tibial insert 20 and a modular lateral tibial insert 30 from a group or supply of available modular medial and lateral tibial inserts 20, 30 in a manner that replaces the stability, range of motion, and kinematics of a natural knee for any given patient, while creating an option for the surgeon to avoid recutting the proximal tibia. For example, if the position of the initial tibial resection is too high, the surgeon may elect to use a medial tibial insert 20 and a lateral tibial insert 30 having a lower minimum thickness MT, LT compared to medial tibial inserts 20 and a lateral tibial inserts 30 having greater minimum thicknesses MT, LT in a supply (i.e., kit). Similarly, if the tibial resection is coplanar with a transverse plane and the surgeon wishes to introduce a varus or valgus angle via the tibial insert and/or an AP slope angle θ via the tibial insert, the surgeon can select a medial tibial insert 20 and a lateral tibial insert 30 from a supply of available medial and lateral tibial inserts 20, 30 having different ML slope angles α and/or AP slope angles θ to achieve the desired joint line without having to recut the tibia.

Upon selection of the desired modular medial insert component 20c and the desired modular lateral insert component 30b, locking fasteners 24a are selected and inserted into the receivers 34 (e.g., holes 34a) that extend into the body 15 of the lateral component 30 through the lateral component inner periphery side 15a and the body 25 of the medial component 20 through the medial component inner periphery side 25a respectively. In this manner, a tibial insert construct 44 can be created. It is contemplated that the assembly and disassembly of the tibial insert construct 44 prior to insertion into the tibial component 35 can be advantageous for trialing the range of motion of the knee before the resected tibia is fully reamed to accommodate the final tibial component 35.

Although two projection receivers locking mechanisms 27 comprising opposing holes 34a and a locking fastener 24a extending into each of the two opposing holes 34a is depicted, it will be appreciated that other exemplary embodiments can comprise fewer or additional sets of opposing holes 34a and a locking fastener 24a extending therebetween.

FIG. 4B is an isometric view of an exemplary modular tibial insert assembly 10 shown in the assembled configuration. Once the surgeon is satisfied with the tibial insert construct 44 (which in the depicted embodiment comprises modular medial insert component 20c and modular lateral insert component 30b), distal receivers 42 (see also FIG. 11B) on the distal side 13, 23 of the tibial insert construct 44 are inserted under the proximal projections 49 on the retaining wall 47 of the tibial baseplate 33 to fixedly engage the tibial insert construct 44 to the tibial baseplate 33. Because of the nature of the incision and access to the operative area, the surgeon typically slides the posterior sides 15d, 25d of the tibial insert construct 44 towards proximal projections 49 in the retaining wall 47 of the posterior side of the tibial baseplate 33 before snapping the distal receivers 42 at the anterior sides 15c, 25c of the tibial insert construct 44 under proximal projections 49 at the anterior side of the tibial baseplate 33 to fixedly engage (and thereby lock or secure) the tibial insert construct 44 to the tibial baseplate 33.

The distal receivers 42 are an example of a distal locking mechanism. It will be appreciated that the modular medial insert component 20 can comprise a medial component distal locking mechanism. Likewise, the modular lateral insert component 30 can comprise a lateral component distal locking mechanism. The proximal projections 49 of the retaining wall 47 is an example of a complementary proximal locking mechanism. A complementary proximal locking mechanism configured to lock with the medial component distal locking mechanism is referred to as a “medial component complementary locking mechanism” of the baseplate of the prior implanted tibial component. Likewise, a complementary proximal locking mechanism configured to lock with the lateral component distal locking mechanism is referred to as a “lateral component complementary locking mechanism” of the baseplate of the prior implanted tibial component. Collectively, these maybe referred to as “distal locking mechanisms.” Although projection-receiver distal locking mechanisms are depicted with reference to FIGS. 4B, 11A, and 11B, other distal locking mechanisms are considered to be within the scope of this disclosure. These include, but are not limited to, magnets of opposite polarity, an adhesive, a bonding agent, a thermal engagement mechanism, or combinations or permutations of the forgoing distal locking mechanisms. Indirect distal locking mechanisms in the manner described supra are also considered to be within the scope of this disclosure.

If a surgeon needs to remove the tibial insert construct 44, the surgeon can insert a key into the anterior keyhole 40 to dislodge the tibial insert construct 44.

Although FIG. 4B depicts the medial articular surface 21 of the selected modular medial insert component 20 as being substantially flush and continuous with the lateral articular surface 31 of the selected modular lateral insert component 30 in the assembled configuration, it will be appreciated that in other exemplary embodiment, the articular surfaces 21, 31 can be discontinuous in the assembled configuration. That is, a step may be created between the adjacently disposed modular medial insert component 20 and the modular lateral insert component 30 in the assembled configuration.

FIG. 5 is an isometric partially exploded view of an exemplary modular tibial insert assembly 10 comprising a modular medial insert component 20, a modular lateral insert component 30, a tibial component 35 comprising a tibial baseplate 33, and a femoral component 45 in a disassembled configuration. The tibial component 35 comprises a keel 56 extending downwardly or distally away from the baseplate 33. Fins 57 typically extend downwardly or distantly away from the baseplate 33 on the medial and lateral sides of the keel 56, respectively. The fins 57 help to stabilize and seat the tibial component 35 when the tibial component 35 is surgically implanted into the resected proximal tibia. The superior surface of the tibial baseplate 33 can further comprise anterior recesses 58, which may provide a secondary distal locking mechanism for secondary anterior distal projections (see 42) extending from the exemplary modular medial insert component 20 and/or modular lateral insert component 30. A posterior baseplate post 59 extends upwardly or superiorly from the baseplate 33 and toward the anterior side. In the depicted embodiment, the posterior baseplate post 59 is disposed at eight degrees from a midline that bisects the tibial component 35 in the sagittal plane. It is contemplated that a posterior baseplate post 59 disposed at between five and eleven degrees from the midline that bisects the tibial component 35 in the sagittal plane can facilitate the insertion and removal of the exemplary modular medial and lateral components 20, 30 in a minimally invasive knee arthroplasty. A minimally invasive knee arthroplasty is typically characterized by using a smaller anteromedial incision of about 7 centimeters to about 11 centimeters to reduce wound size and facilitate patient recovery compared to a standard arthroplasty, which is typically characterized an anterior incision of about 15 centimeters to about 26 centimeters expose the entire knee joint and to permit the subluxation and inversion of the patella.

Although a tibial component 35 configured for use in a TKA is depicted, it will be appreciated that a tibial component configured for a unicondylar knee arthroplasty is considered to be within the scope of this disclosure. A unicondylar tibial component comprises a tibial baseplate 33 that is dimensions to be disposed over either the resected medial portion of the tibia or the resected lateral portion of the tibia. It will be appreciated that in such exemplary unicondylar embodiments, only a modular medial insert component 20 or only a modular lateral insert component 30 will be engaged to the unicondylar tibial baseplate 33 as needed. That is, in a medial partial knee arthroplasty, a modular medial insert component 20 selected from a supply of possible modular medial insert components will be placed on the tibial baseplate 33 of a medial unicondylar tibial component 35. Likewise, in a lateral partial knee arthroplasty, a modular lateral insert component 30 selected from a supply of possible modular lateral insert components 30 will be placed on the tibial baseplate 33 of a lateral unicondylar component 35.

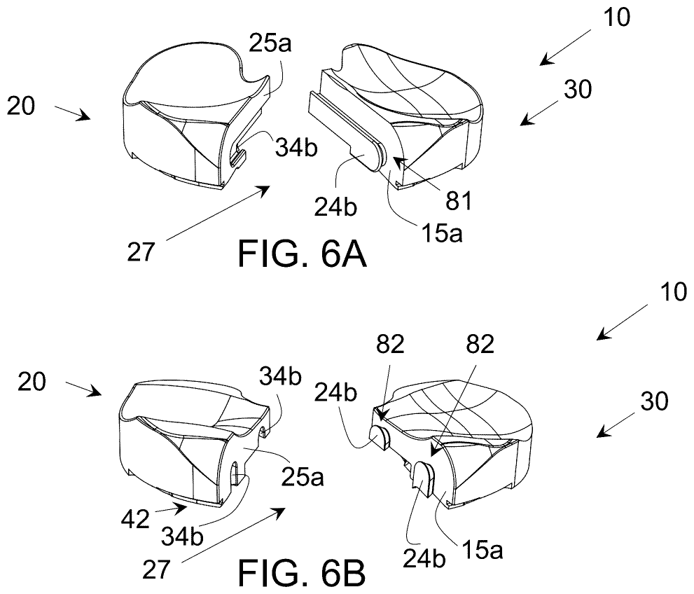

FIG. 6A is an isometric view of an exemplary modular tibial insert assembly 10 shown in the disassembled configuration, wherein the locking mechanism 27 is a dovetail locking mechanism. The projection 24 is a dovetail projection 24b depicted as extending from the lateral component inner periphery side 15a. The dovetail projection 24b is disposed generally horizontally in the anterior-posterior direction and has a rounded anterior end 81 to prevent impingement when sliding into the receiver 34 disposed in the medial component inner periphery side 25a. In the depicted embodiment, the receiver 34 is a slot 34b that is disposed generally horizontally in the anterior-posterior direction and is closely dimensioned to accommodate the dovetail projection 24b extending from the modular lateral insert component 30 in an assembled configuration. The depicted dovetail projection 24b extends to the lateral component posterior periphery side 15d. In other exemplary embodiments, the posterior end of the dovetail projection 24b may terminate before reaching the lateral component posterior periphery side 15d. In still other exemplary embodiments, portions of the body of the dovetail projection 24b may be removed at intervals to reduce weight or material without substantially compromising the function of the locking mechanism 27.

It will be appreciated that in exemplary embodiments comprising projection-receiver locking mechanisms 27, a projection 24 may extend from either the modular lateral insert component 30, or the modular medial insert component 20. Likewise, a complementary receiver 34 that closely receives the projection 24 may be disposed in either the modular lateral insert component 30, or the modular medial insert component 20 provided that the complementary receiver 34 closely receives the projection 24 in the assembled configuration. Similarly, embodiments comprising multiple projections 24 and multiple complementary receivers 34 are considered to be within the scope of this disclosure. Combinations of the forgoing are considered to be within the scope of this disclosure.

Without being bound by theory, it is contemplated that a sliding dovetail locking mechanism 27 may permit selective assembly and disassembly of the exemplary modular tibial insert assembly 10 in less time than with the locking assembly depicted in FIGS. 1-5, thereby permitting faster testing of knee kinematics when surgically implanted into the patient. It is contemplated that multiple tibial insert constructs 44 can be provided to the surgeon, wherein one or more of the provided multiple tibial insert constructs 44 comprises a modular medial insert component 20 or a modular lateral insert component 30 that comprises a specification having a value that differs from the value of the specification of a modular medial insert component 20 or a modular lateral insert component 30 of another provided tibial insert construct 44. The specifications may be selected from the group consisting essentially of: an AP slope, an ML slope, a minimum thickness, a dwell point location, a geometry of an articular surface, a radius of curvature, a size, and a biocompatible material. In other exemplary embodiments, a given specification (e.g., the AP slope) in a set of provided tibial insert constructs 44 can differ from the value of every other given specification in the provided set of tibial insert constructs 44.

FIG. 6B is an isometric view of an exemplary modular tibial insert assembly 10 shown in the disassembled configuration, wherein the locking mechanism 27 comprises multiple dovetail locking mechanisms. The projections 24 are dovetail projections 24b extending from the lateral component inner periphery side 15a. The multiple dovetail projections 24b are disposed generally vertically in the superior-inferior direction and have a rounded superior end 82 to prevent impingement when sliding into complementary multiple receivers 34 disposed in the medial component inner periphery side 25a. In the depicted embodiment, each of the multiple receivers 34 is a slot 34b that is disposed generally vertically in the superior-inferior direction and is closely dimensioned to accommodate an adjacent dovetail projection 24b of the multiple dovetail projections 24b extending from the modular lateral insert component 30 in an assembled configuration. In the depicted embodiment, the distal ends of the multiple dovetail projections 24b and the multiple slots 34b extend to the distal sides 15b, 25b of the modular lateral insert component 30 and the modular medial insert component 20, respectively. In such embodiments, the profile of the distal ends of the dovetail projections 24b and the slots 34b desirably conform to the profile of the distal sides 23, 13 of the modular medial insert component 20 and the modular lateral insert component 30 respectively to accommodate the profile of a distal locking mechanism that is configured to engage the modular inserts 20, 30 to the tibial baseplate in the assembled configuration.

Examples of other types of projection-receiver locking mechanisms that are considered to be within the scope of this disclosure include, but are not limited to, clamps, hooks, lips, recesses, tongues and grooves, dovetail rails, complementary flanges, other complementary mechanical engagement mechanisms, and combinations thereof.

It will further be appreciated that in other exemplary embodiments an intermediary structure (e.g., a linking baseplate) can mechanically engage the modular medial insert component 20 to the modular lateral insert component 30 in an engaged configuration. In still other exemplary embodiments, multiple intermediary structures can mechanically engage the modular medial insert component 20 to the modular lateral insert component 30 in an engaged configuration.

In still other exemplary embodiments, the modular medial tibial insert component 20 and the modular lateral insert component 30 can comprise a groove that traverses the periphery of the medial tibial insert component 20 and the lateral tibial insert component 30. A band can be closely fitted into the peripheral groove in an engaged configuration. In other embodiments, the peripheral groove can be disposed in the retaining lip of the baseplate 33 of the tibial component 35 and a retaining band may be disposed between the peripheral groove of the modular medial and lateral insert components 20, 30 and the peripheral groove of the retaining lip. In other exemplary embodiments, the retaining band may be absent and peripheral projections from the modular medial and lateral insert components 20, 30 extend into the peripheral groove of the retaining lip in an engaged configuration. All selective mechanical engagement mechanisms are considered to be within the scope of this disclosure.

The modular medial and/or lateral components 20, 30 of the modular tibial insert assembly 10 can be made from a variety of possible materials. These materials include biocompatible polyethylene (“PE”). Ultra-high molecular weight polyethylene (“UHMWPE”), especially highly crosslinked UHMWPE may be desirable because such materials have been demonstrated to have improved material fatigue resistance after repeated use. In some exemplary embodiments, the UHMWPE can be infused with Vitamin E to further stabilize the UHMWPE and mitigate oxidative degradation of the UHMWPE. Other materials include polyether ether ketone (“PEEK”), which is resistant to mechanical and thermal degradation while being radiolucent and hydrophobic, thereby mitigating the risk of bone ingrowth.

Although FIGS. 1-6B depict the modular medial and lateral tibial insert components 20, 30 as being fixedly engaged to each other in the assembled configuration to form a tibial insert construct 44 prior to being locked into the tibial base component 35, nothing in this disclosure limits the invention to these depicted embodiments. For example, in the embodiments depicted with reference to FIGS. 11A and 11B, the tibial insert construct 44 is formed when the modular medial and lateral tibial insert components 20, 30 are fixedly engaged to the tibial component 35 in the assembled configuration. In other exemplary embodiments, the medial insert component 20 can be fixedly engaged to the tibial base component 35 and the lateral inset component 30 can move (e.g., translate (e.g., slide), rotate, pivot, or combinations thereof) relative to the tibial base component 35 in the assembled configuration, or vice versa. In other exemplary embodiments, the medial and lateral insert components 20, 30 can be engaged to each other in an engaged configuration but not entirely fixedly engaged to the tibial base component 35 in the assembled configuration, such that the engaged medial and lateral insert construct can move (e.g., translate (e.g., slide), rotate, pivot, or combinations thereof) relative to the tibial base component 35 in the assembled configuration.

Furthermore, although the embodiments depicted in FIGS. 1-6B relate to exemplary modular tibial insert assemblies 10 that are designed to be inserted into a total knee endoprosthesis for use over the life of the implant, nothing in this disclosure limits the exemplary modular tibial insert assemblies 10 to be modular tibial insert assembly 10 than can only be used with the final, surgically implanted endoprosthesis. In other exemplary embodiments, the modular tibial insert assembly 10 can be a trial modular tibial insert assembly 10. It will be understood that trial medial and lateral insert components 20, 30 generally have the same functional dimensions as final endoprosthetic medial and lateral insert components 20, 30, but the trial medial and lateral insert components 20, 30 may be made from less robust (but still biocompatible) materials, may be disposable, or may be configured to be assembled and disassembled with even greater ease than the final endoprosthetic medial and lateral insert components 20, 30 because the trial medial and lateral insert components 20, 30 do not have to be designed to withstand prolonged use and wear forces.

FIG. 7A is a top down view of an exemplary lateral insert component 30 having an anterior-posterior (“AP”) slope angle θ and a dovetail projection 24b extending from the lateral component inner periphery side 15a. The lateral component articular surface 31 is viewed from the proximal side 38 (e.g., a lateral insert component first side).

FIG. 7B is a cross-sectional side view of the exemplary lateral insert component 30 of FIG. 7A taken along the line A-A. The lateral insert component 30 comprises an AP slope angle θ. The AP slope angle θ is defined by the angle formed between a hypothetical line E extending in the AP direction in the transverse and sagittal planes along the distal side 13 of the lateral insert component 30 and the hypothetical line F extending tangent to the lowest point of the lateral component articular surface 31. In a medial pivot exemplary embodiment, the lateral component articular surface 31 desirably comprises a toroid shape. In such embodiments, the hypothetical line F extends tangent to the lowest point of the lowest medial-lateral curve defining the toroid shape. Likewise, for a medial insert component 20, the AP slope angle θ is defined by the angle formed between a hypothetical line E extending in the AP direction in the transverse and sagittal planes along the distal side 23 of the medial insert component 20 and the hypothetical line F extending tangent to the lowest point of the medial component articular surface 21.

The AP slope angle θ of the lateral insert component 30 in the depicted embodiment is 6 degrees. It is contemplated that in certain exemplary embodiment, modular medial insert components 20 or modular lateral insert components 30 may be provided having an AP slope angle θ in a range of −5 degrees to 15 degrees. In practice, it will be appreciated that the total AP travel angle of a medial pivot knee joint is a resultant angle formed by the AP angle of the tibial baseplate 33 (if present), the AP slope angle θ of the lateral insert component 30 and the medial pivot angle.

FIG. 8A is a top down view of an exemplary lateral insert component 30 having a medial-lateral (“ML”) slope Δ and a dovetail projection 24b extending from the lateral component inner periphery side 15a. The lateral component articular surface 31 is viewed from the proximal side 38 (e.g., a lateral insert component first side).

FIG. 8B is a cross-sectional side view of the exemplary lateral insert component 30 of FIG. 8A taken along the line B-B. The lateral insert component 30 comprises an ML slope angle Δ. The ML slope angle Δ is defined by the angle formed between a hypothetical line G extending in the ML direction in the transverse and sagittal planes along the distal side 13 of the lateral insert component 30 and the hypothetical line H extending tangent to the lowest point of the lateral component articular surface 31. In a medial pivot exemplary embodiment, the lateral component articular surface 31 desirably comprises a toroid shape. In such embodiments, the hypothetical line G extends tangent to the lowest point of the lowest curve defining the toroid shape. Likewise, for a medial insert component 20, the ML slope angle Δ is defined by the angle formed between a hypothetical line G extending in the ML direction in the transverse and sagittal planes along the distal side 23 of the medial insert component 20 and the hypothetical line H extending tangent to the lowest point of the medial component articular surface 21.

The ML slope angle Δ of the lateral insert component 30 in the depicted embodiment is 6 degrees. It is contemplated that in certain exemplary embodiment, modular medial insert components 20 or modular lateral insert components 30 may be provided having an ML slope angle Δ in a range of −5 degrees to 15 degrees. In practice, it will be appreciated that the total MP travel angle of a medial pivot knee joint is a resultant angle formed by the varus/valgus angle of the tibial baseplate 33 (if present), the ML slope angle Δ of the lateral component insert 20, and the medial pivot angle.

FIG. 9A is a top down view of an exemplary medial insert component 20, wherein the proximal side 28 comprises a medial pivot articular surface 21 having a concave portion 22.

FIG. 9B is a cross-sectional side view of the exemplary medial insert component 20 of FIG. 9A taken along the line C-C. FIG. 9B more clearly depicts the dwell point 93 disposed at the nadir of the concave portion 22. In certain exemplary embodiments, the dwell point 93 is disposed between 39% to 45% of the AP length mAP of the medial insert component 20 and desirably between 41% to 43% of the AP length of the medial insert component 20 as measured from the posterior side 25d. Without being bound by theory, it is contemplated that a dwell point 93 disposed in this position for an appropriately sized knee can closely approximate the pivoting point of the natural knee. A radius of curvature of the medial component articular surface 21 can be defined by the curve formed by a radius (see mpVA) extending towards a center of flexion-extension rotation mR of the medial femoral condyle to the articular surface 21. In an exemplary embodiment, the radius of curvature has a radius that comprises the radial distance (see mpVA) from the center of flexion-extension rotation mR of the medial femoral condyle 48a (FIG. 5) to the articular surface 21 of the medial tibial insert component 20. In another exemplary embodiment, the radius of curvature has a radius that is 30 percent (“%”) to 50% of the radial distance (see mpVA) from the center of flexion-extension rotation mR of the medial femoral condyle to the articular surface 21 of the medial tibial insert component 20. In another exemplary embodiment, the radius of curvature has a radius that is 5% to 15% of the radial distance (see mpVA) from the center of flexion-extension rotation mR of the medial femoral condyle to the articular surface 21 of the medial tibial insert component 20. In one exemplary embodiment, the medial pivot articular surface 21 of the medial tibial insert component 20 comprises a constant radius of curvature. In other exemplary embodiments, the medial pivot articular surface 21 can comprise a first radius of curvature C1 extending from the dwell point 93 to the anterior side 25c and a second radius of curvature C2 extending from the dwell point 93 to the posterior side 25d, wherein the radius R1 of the first radius of curvature C1 is within 5% to 15% of the radial distance (see mpVA) from the center of flexion-extension rotation mR of the medial femoral condyle to the articular surface 21 of the medial tibial insert component 20, and the radius R2 of the second radius of curvature C2 is between 30% to 50% of the radial distance (see mpVA) from the center of flexion-extension rotation mR of the medial femoral condyle to the articular surface 21 of the medial tibial insert component 20. Without being bound by theory, it is contemplated that two radii of curvature in the manner described herein can permit medial condyle rollback (i.e., the rotation and sliding of the medial femoral condyle 48a posteriorly along the medial component articular surface 21) in deep flexion while preserving desired stability and medial pivot rotation in normal ambulatory movement.

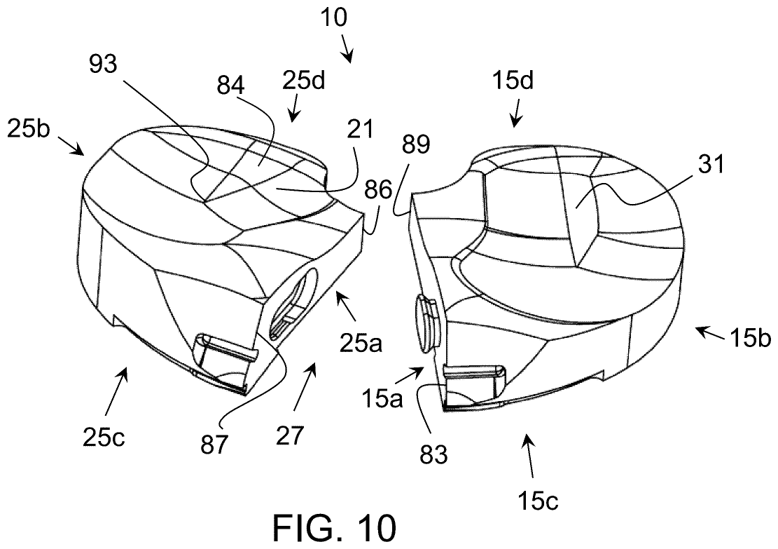

FIG. 10 is an anterior perspective view of an exemplary medial insert component 20 and an exemplary lateral insert component 30 comprising a dovetail locking mechanism 27, wherein the articular surface 21 of the medial insert component 20 comprises a portion having a triangular flat surface 84, such as the triangular flat surface 84 disposed in U.S. Pat. No. 7,261,740 to Tuttle et. al., the entirety of which is incorporated herein by reference. Without being bound by theory, it is contemplated that the triangular flat surface 84 allows the medial femoral condyle 48a (FIG. 5) to slide and rotate posteriorly during deep knee flexion.

FIG. 11A is an anterior and partially superior facing view of an exemplary modular tibial insert assembly 10 comprising a modular medial insert component 20, a modular lateral insert component 30, and a tibial component 35 in a disassembled configuration, wherein the medial insert component 20 and the lateral insert component 30 are configured to engage the tibial baseplate 33 of the tibial component 35 without also mechanically engaging each other.

FIG. 11B is an anterior and partially inferior facing view of the exemplary modular tibial insert assembly 10 of FIG. 11A shown in a partially assembled configuration.

In practice, a surgeon or technician may select a modular medial insert component 20 and a modular lateral insert component 30 from a supply (e.g., kit or surgical tray) of multiple modular medial insert components 20 wherein the modular medial insert components 20 in the supply have specifications that differ from specifications of other modular medial insert components 20 in the supply. Once a desired modular medial insert component 20 is selected, the surgeon can then slide a posterior distal receiver 42d on the posterior side 25d of the modular medial insert component 20 under a proximal projection 49 projecting from the retaining wall 47 of the tibial baseplate 33 and then snap the anterior distal receiver 42c under an anterior proximal projection 49a projecting from the retaining wall 47 of the tibial baseplate 33 to thereby insert and fixedly engage the exemplary modular medial insert component 20 onto the tibial baseplate 33. FIG. 11A further depicts inner distal receivers 42a on the medial insert component 20 and the lateral insert component 30. These inner distal receivers 42a likewise slide under a proximal projection 49 extending transversely from the posterior baseplate post 59 to thereby insert and fixedly engage (or to thereby lock) the exemplary modular medial insert component 20 or the exemplary modular lateral insert component 30 onto the tibial baseplate 33 in the assembled configuration. When only the modular medial insert component 20 or the modular lateral insert component 30 are so inserted, the modular tibial insert assembly 10 can be said to be shown in a “partially assembled configuration.”

Likewise, a surgeon or technician may select a modular lateral insert component 30 from a supply (e.g., kit or surgical tray) of multiple lateral insert components 30 wherein the modular lateral insert components 30 in the supply have specifications that differ from specifications of other modular lateral insert components 30 in the supply. Once a desired modular lateral insert component 30 is selected, the surgeon can then slide a posterior distal receiver 42d on the posterior side 15d of the modular lateral insert component 30 under a proximal projection 49 projecting from the retaining wall 47 of the tibial baseplate 33 and then snap the anterior distal receiver 42c under an anterior proximal projection 49a projecting from the retaining wall 47 of the tibial baseplate 33 to thereby insert and fixedly engage the exemplary modular lateral insert component 30 onto the tibial baseplate 33. When both the modular medial insert component 20 and the modular lateral insert component 30 are so inserted, the modular tibial insert assembly 10 can be said to be in an “assembled configuration.”

It will be appreciated that in the exemplary embodiment depicted in FIGS. 11A and 11B, the tibial insert construct 44 exists when the exemplary modular tibial insert assembly 10 is in the assembled configuration.

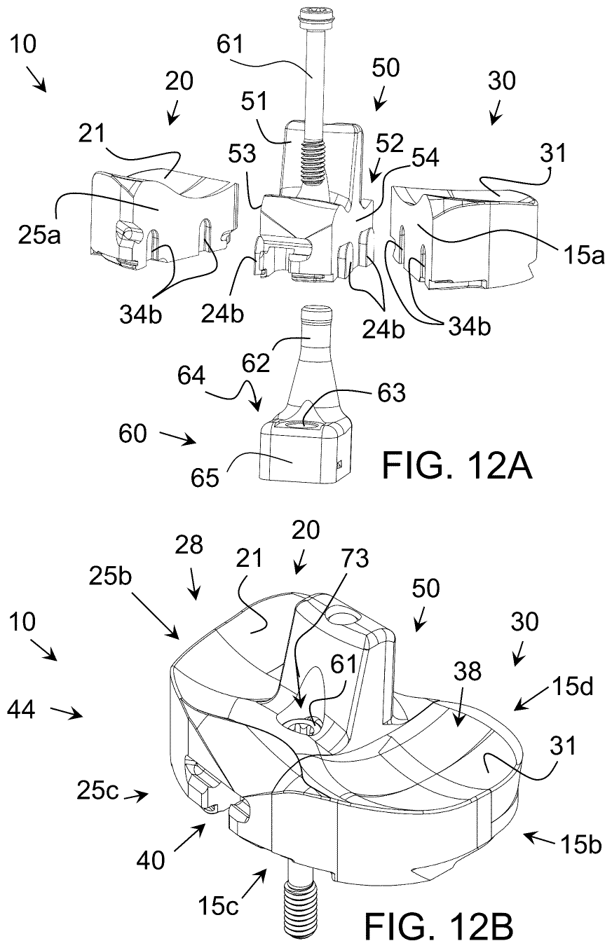

FIG. 12A is an anterior exploded view of an exemplary CCK type modular tibial insert assembly 10 shown in a disassembled configuration. An exemplary modular medial insert component 20 and a modular lateral insert component 30 are provided consistent with the exemplary embodiments described supra. With respect to embodiments that comprise additional, modular tibial insert components, the modular medial tibial insert component 20 may alternatively be referred to as a “first modular tibial insert component” and the modular lateral tibial insert component 30 may alternatively be referred to as a “second modular tibial insert component” or vice versa. FIGS. 12A and 12B depict a third modular tibial insert component 50 configured to be to be disposed between and fixedly engaged to the first tibial insert component 20 and the second tibial insert component 30 in the assembled configuration.

In the depicted embodiment, the third modular tibial insert component 50 is a CCK modular tibial insert component. The depicted CCK modular tibial insert component 50 comprises a post 51 extending superiorly from the superior side 52 of the CCK modular tibial insert component 50. Flat medial and lateral post surfaces 53, 54 bound the medial and lateral sides of the CCK modular tibial insert component 50. The generally rectangular shape of the post and the flat medial and lateral post surfaces 53, 54 reduce internal and external rotation of the knee around a generally vertical axis when the assembly 10 is in the assembled and implanted configuration. The third modular tibial insert component 50 is configured to fixedly engage the first and second modular tibial insert components 20, 30 in any of the manners described supra.

It will be appreciated that in other exemplary embodiments, the third modular tibial insert component 50 can be a PS modular tibial insert component, a CR modular tibial insert component, or a CS modular tibial insert component.

FIG. 12A shows dovetail projections 24b extending from the flat medial post surface 53 and the flat lateral post surface 54 respectively to be closely received by closely dimensioned and complementary slots 34b extending into the medial component inner periphery side 25a and the lateral component inner periphery side 15a, respectively. Exemplary third modular tibial insert components 50 can be provided in a kit or other supply in the manner described elsewhere herein and can have a specification that differs from other specifications of third modular tibial insert components 50 in the kit or supply.

The depicted assembly 10 further comprises a fourth tibial insert component 60, wherein the fourth tibial insert component 60 is a modular stabilizing post component 60 configured to be disposed between the third tibial insert component 50 and a baseplate 33 of a tibial component 35 in an assembled and implanted configuration. The modular stabilizing post component 60 comprises a stabilizing post 62 extending superiorly from a superior side 64 of the modular stabilizing post component 60 and a faster opening 63 extending generally superiorly to inferiorly in an anterior portion 65 of the modular stabilizing post component 60. The modular stabilizing post component 60 is typically made from cobalt chrome, titanium, or other durable biocompatible metal. The third tibial insert component 50 is typically make from one of the polymer materials described above or from a ceramic.

FIG. 12B is an anterior and partially inferior facing perspective view of the exemplary CCK modular tibial insert assembly 10 of FIG. 12A shown in an assembled and implanted configuration. In the assembled configuration, a fastener 61 extends superiorly-inferiorly through an insert fastener opening 73 that is disposed over and aligned with the fastener opening 63 of the modular stabilizing post component 60. The distal end of the fastener is desirably configured to be inserted into and engage the tibial component 35. In the depicted embodiment, this is achieved with screw threads, but other affixing mechanisms known to those having ordinary skill in the art are considered to be within the scope of this disclosure. The resulting tibial insert construct 44 can then be affixed to the baseplate 33 of the tibial component 35.

Exemplary kits and assemblies may permit less resection, reconstruction of pre-diseased varus or valgus angle using combinations of medial and lateral components of different thicknesses relative to each other. Medial and lateral components with built in anterior-posterior slope in some embodiments. In other exemplary embodiments, anterior-posterior slope built into tibial component such as the one described in U.S. Pat. Pub. No. 2023/015783 to Harris, Jr., the entirety of which is incorporated herein by reference.

The exemplary modular medial insert components 20, modular lateral insert components 30, third modular tibial insert components 50, and fourth modular tibial insert components 60 of the exemplary modular tibial insert assembly 10 described herein can be provided in the form of a kit. A kit can constitute a supply of modular medial insert components, modular lateral insert components 30, third modular tibial insert components 50, and/or fourth modular tibial insert components 60. It will be appreciated that the modular medial insert components 20, the modular lateral insert components 30, the third modular tibial insert components 50, and the fourth modular tibial insert components 60 can be provided in different sizes. Chiral components can be provided for a left knee and a right knee.

The modular medial insert components 20, modular lateral insert components 30, third modular tibial insert components 50, and fourth modular tibial insert components 60 can be provided with a number of different specifications (i.e., with a specification that differs from at least one other specification in the supply), including different sizes, thicknesses, physical materials, modular insert to modular insert locking mechanism, modular insert to tibial baseplate engagement mechanisms, or combinations of any of the foregoing. Furthermore, with regard to the modular medial insert components 20 and modular lateral insert components 30 in particular, the exemplary modular medial insert components 20 and modular lateral insert components 30 can be provided with a number of further different specifications (i.e., with a specification that differs from at least one other specification in the supply), including ML slope angles, AP slope angles, articular surfaces, radii of curvature, dwell points, concavity, convexity, modular insert to modular insert locking mechanism, modular insert to tibial baseplate engagement mechanisms, or combinations of any of the foregoing. The components of the kit are preferably arranged in a convenient format, such as in a surgical tray or case. However, the kit components do not have to be packaged or delivered together, provided that they are assembled or collected together in the operating room for use at the time of surgery.

An exemplary kit can include any suitable embodiment of a modular medial insert component 20, variations of the exemplary modular medial insert components 20 described herein, and any other exemplary modular medial insert components 20 according to an embodiment. While it is contemplated that an exemplary kit may further include one or more tibial base components, it will be appreciated that certain kits may lack some or all of these components.

Any suitable embodiment of a modular lateral insert component 30, variations of the modular lateral insert components 30 described herein, and any other modular lateral insert components 30 according to an embodiment are considered to be within the scope of this disclosure.

Any suitable embodiment of a third modular insert component 50, variations of the third modular insert components 50 described herein, and any other third modular insert components 50 according to an embodiment are considered to be within the scope of this disclosure.

Any suitable embodiment of a fourth modular insert component 60, variations of the fourth modular insert components 60 described herein, and any other fourth modular insert components 40 according to an embodiment are considered to be within the scope of this disclosure. Selection of a suitable number or type of modular medial insert components 20 and modular lateral insert components 30 to include in a kit according to a particular embodiment can be based on various considerations, such as the procedure intended to be performed using the components included in the kit.

Whereas much of the preceding detailed description described a modular tibial insert assembly 10, it will be appreciated that tibial insert comprising a medial component and a lateral component that further comprise specifications that are independently assessed and selected based on the anatomy of a patient or a state of a prior implanted endoprosthetic implant are considered to be within the scope of this disclosure.

In such exemplary embodiments, the tibial insert can comprise a single, unitary piece, but the specifications of the medial portion and lateral portion of the exemplary tibial insert can be independently assessed and selected. In one such exemplary embodiment, it is contemplated that such an exemplary tibial insert can be manufactured in advance of the surgical procedure based on pre-operative imaging of the patient's anatomy and/or based upon a review of the patient's medical or surgical history. In another such exemplary embodiment, it is contemplated that such an exemplary tibial insert can be produced during the scheduled operation time based on the surgeon's assessment of the knee joint, which may be further informed by interoperative measurement or assessment instrumentation. Once the desired specifications of the medial portion and the lateral portion have been assessed and selected, it is contemplated that these selection can be transmitted to a manufacturing device, such as an additive manufacturing device or a milling manufacturing device, to produce a tibial insert with the desired specifications. A sterilizer, such as an ethylene oxide, (“EtO”) sterilizer, is desirably provided to sterilize the manufactured tibial insert prior to surgical implantation. It is contemplated that such a system may reduce the amount of available inventory that is deemed necessary to support knee arthroplasties. Instead of supplying a surgical tray or kit comprising a number of components with different specifications, fewer (and in some cases a single) tibial insert can be produced to the desired specifications.

An exemplary modular tibial insert assembly comprises: a modular medial insert component, the modular medial insert component having a medial component proximal side proximally disposed from a medial component distal base side, wherein a medial component body extends between the medial component proximal side and the medial component distal base side; a modular lateral insert component, the modular lateral insert component having a lateral component proximal side proximally disposed from a lateral component distal base side, wherein a lateral component body extends between the lateral component proximal side and the lateral component distal base side; wherein the modular tibial insert assembly has an assembled configuration and a disassembled configuration, and wherein the modular medial insert component is configured to fixedly engage the modular lateral insert component in the assembled configuration.

In an exemplary embodiment, the medial component proximal side of the modular tibial insert assembly further comprises a medial component articular surface, wherein the medial component articular surface is a medial pivot medial component articular surface.

In an exemplary embodiment, the lateral component proximal side of the modular tibial insert assembly further comprises a lateral component articular surface, wherein the lateral component articular surface is a medial pivot lateral component articular surface.

In an exemplary embodiment, the medial component body further comprises a medial component outer periphery side and a medial component inner periphery side. Such an exemplary embodiment may optionally further comprise the lateral component body further having a lateral component outer periphery side and a lateral component inner periphery side. Such an exemplary embodiment may optionally still further comprise the medial component inner periphery side being configured to mechanically engage the lateral component inner periphery side in the assembled configuration.

In an exemplary embodiment, the lateral component body further comprises a lateral component outer periphery side and a lateral component inner periphery side.

In an exemplary embodiment, the medial component body and the lateral component body further comprise a locking mechanism selected from the group consisting essentially of: a projection-receiver engagement mechanism, a magnetic engagement mechanism, a thermal engagement mechanism, and an adhesive engagement mechanism.

In an exemplary embodiment, the modular tibial insert assembly further comprises a plurality of modular medial insert components, the plurality of modular medial insert components comprising a first modular medial insert component and a second modular medial insert component, the first modular medial insert component being of a different size than the second modular medial insert component, wherein the modular medial insert component is selected from the plurality of modular medial insert components.

In an exemplary embodiment, the modular tibial insert assembly further comprises a plurality of modular lateral insert components, the plurality of modular lateral insert components comprising a first modular lateral insert component and a second modular lateral insert component, the first modular lateral insert component being of a different size than the second modular lateral insert component, wherein the modular lateral insert component is selected from the plurality of modular lateral insert components.

In an exemplary embodiment, the modular tibial insert assembly further comprises a tibial base component, the tibial base component comprising a tibial baseplate. In such an embodiment comprising a tibial baseplate, the modular medial component distal base side is optionally configured to fixedly engage the tibial baseplate. In such an embodiment comprising a tibial baseplate, the modular lateral component distal base side is optionally configured to fixedly engage the tibial baseplate.

In an exemplary embodiment, the modular medial insert component comprises a biocompatible material selected from the group consisting essentially of: a polyethylene, a polyether ether ketone, and a ceramic.

In an exemplary embodiment, the modular lateral insert component comprises a biocompatible material selected from the group consisting essentially of: a polyethylene, a polyether ether ketone, and a ceramic.

In an exemplary embodiment, the medial component body of the modular tibial insert assembly further comprises a medial component outer periphery side and a medial component inner periphery side, the lateral component body further comprises a lateral component outer periphery side and a lateral component inner periphery side, and the medial component inner periphery side physically abuts the lateral component inner periphery side in the assembled configuration. In such an embodiment, a distal edge of the medial component outer periphery side optionally abuts a medial peripheral edge of a tibial base component, and a distal edge of the lateral component outer periphery side optionally abuts a lateral peripheral edge of the tibial base component in the assembled configuration.

In an exemplary embodiment, the modular tibial insert assembly further comprises a plurality of modular medial insert components, the plurality of modular medial insert components comprising a first modular medial insert component and a second modular medial insert component, the first modular medial insert component having a different specification than the second modular medial insert component, wherein the specification is selected from the group consisting essentially of: an AP slope angle, an ML slope angle, a minimum thickness, a dwell point location, an articular surface, a radius of curvature, and a biocompatible material.

In an exemplary embodiment, the modular tibial insert assembly further comprises a plurality of modular lateral insert components, the plurality of modular lateral insert components comprising a first modular lateral insert component and a second modular lateral insert component, the first modular lateral insert component having a different specification than the second modular lateral insert component, wherein the specification is selected from the group consisting essentially of: an AP slope angle, an ML slope angle, a minimum thickness, an articular surface, and a biocompatible material.

An exemplary endoprosthetic knee implant modular tibial insert assembly comprises: a modular medial tibial insert component, the modular medial tibial insert component having a medial component proximal side proximally disposed from a medial component distal base side, wherein a medial component body extends between the medial component proximal side and the medial component distal base side; a modular lateral tibial insert component, the modular lateral tibial insert component having a lateral component proximal side proximally disposed from a lateral component distal base side, wherein a lateral component body extends between the lateral component proximal side and the lateral component distal base side; a tibial component comprising a tibial baseplate, the tibial baseplate comprising a superior surface; wherein the endoprosthetic knee implant modular tibial insert assembly has an assembled configuration and a disassembled configuration, and wherein the modular medial tibial insert component is configured to fixedly engage the tibial component in the assembled configuration.

In such an embodiment, the medial component distal base side optionally further comprises a locking mechanism selected from the group consisting essentially of: a projection-receiver engagement mechanism, a magnetic engagement mechanism, a thermal engagement mechanism, and an adhesive engagement mechanism. In such an embodiment, the superior surface of the tibial baseplate optionally further comprises a complementary locking mechanism selected from the group consisting essentially of: a projection-receiver engagement mechanism, a magnetic engagement mechanism, a thermal engagement mechanism, and an adhesive engagement mechanism, wherein the complementary locking mechanism is configured to fixedly engage the locking mechanism of the medial component distal base side in the assembled configuration of the endoprosthetic knee implant modular tibial insert assembly.

In an exemplary embodiment of the endoprosthetic knee implant modular tibial insert assembly, the medial component proximal side further comprises a medial component articular surface, and wherein the medial component articular surface is a medial pivot medial component articular surface.

In an exemplary embodiment of the endoprosthetic knee implant modular tibial insert assembly, the lateral component proximal side further comprises a lateral component articular surface, and wherein the lateral component articular surface is a medial pivot lateral component articular surface.

In an exemplary embodiment of the endoprosthetic knee implant modular tibial insert assembly, the medial component body optionally further comprises a medial component outer periphery side and a medial component inner periphery side. In such an exemplary embodiment, the lateral component body optionally further comprises a lateral component outer periphery side and a lateral component inner periphery side. In such an exemplary embodiment, the medial component inner periphery side is optionally configured to mechanically engage the lateral component inner periphery side in the assembled configuration.

In an exemplary embodiment of the endoprosthetic knee implant modular tibial insert assembly, the lateral component body optionally further comprises a lateral component outer periphery side and a lateral component inner periphery side.

In an exemplary embodiment of the endoprosthetic knee implant modular tibial insert assembly, the medial component body and the lateral component body optionally further comprise a locking mechanism selected from the group consisting essentially of: a projection-receiver engagement mechanism, a magnetic engagement mechanism, a thermal engagement mechanism, and an adhesive engagement mechanism.

In an exemplary embodiment of the endoprosthetic knee implant modular tibial insert assembly, the modular lateral component distal base side is optionally configured to fixedly engage the tibial baseplate.

In an exemplary embodiment of the endoprosthetic knee implant modular tibial insert assembly, the modular medial insert component optionally comprises a biocompatible material selected from the group consisting essentially of: a polyethylene, a polyether ether ketone, and a ceramic.

In an exemplary embodiment of the endoprosthetic knee implant modular tibial insert assembly, the modular lateral insert component optionally comprises a biocompatible material selected from the group consisting essentially of: a polyethylene, a polyether ether ketone, and a ceramic.

In an exemplary embodiment of the endoprosthetic knee implant modular tibial insert assembly, the medial component body optionally further comprises a medial component outer periphery side and a medial component inner periphery side, the lateral component body further comprises a lateral component outer periphery side and a lateral component inner periphery side, wherein the medial component inner periphery side physically abuts the lateral component inner periphery side in the assembled configuration. In such an embodiment, a distal edge of the medial component outer periphery side optionally abuts a medial peripheral edge of a tibial base component, and a distal edge of the lateral component outer periphery side optionally abuts a lateral peripheral edge of the tibial base component in the assembled configuration.

An exemplary embodiment of the endoprosthetic knee implant modular tibial insert assembly optionally further comprises a plurality of modular medial insert components, the plurality of modular medial insert components comprising a first modular medial insert component and a second modular medial insert component, the first modular medial insert component having a different specification than the second modular medial insert component, wherein the specification is selected from the group consisting essentially of: an AP slope angle, an ML slope angle, a minimum thickness, a size, a dwell point location, an articular surface, a radius of curvature, and a biocompatible material.

An exemplary embodiment of the endoprosthetic knee implant modular tibial insert assembly optionally further comprises a plurality of modular lateral insert components, the plurality of modular lateral insert components comprising a first modular lateral insert component and a second modular lateral insert component, the first modular lateral insert component having a different specification than the second modular lateral insert component, wherein the specification is selected from the group consisting essentially of: an AP slope angle, an ML slope angle, a minimum thickness, a size, an articular surface, and a biocompatible material.

An exemplary endoprosthetic knee implant modular tibial insert assembly comprises: a modular medial tibial insert component, the modular medial tibial insert component having a medial component proximal side proximally disposed from a medial component distal base side, wherein a medial component body extends between the medial component proximal side and the medial component distal base side; a modular lateral tibial insert component, the modular lateral tibial insert component having a lateral component proximal side proximally disposed from a lateral component distal base side, wherein a lateral component body extends between the lateral component proximal side and the lateral component distal base side; a tibial component comprising a tibial baseplate, the tibial baseplate comprising a superior surface; wherein the modular tibial insert assembly has an assembled configuration and a disassembled configuration, and wherein the modular lateral tibial insert component is configured to fixedly engage the tibial component in the assembled configuration.

In an exemplary embodiment of the endoprosthetic knee implant modular tibial insert assembly, the medial component distal base side optionally further comprises a locking mechanism selected from the group consisting essentially of: a projection-receiver engagement mechanism, a magnetic engagement mechanism, a thermal engagement mechanism, and an adhesive engagement mechanism. In such an exemplary embodiment, the superior surface of the tibial baseplate optionally further comprises a complementary locking mechanism selected from the group consisting essentially of: a projection-receiver engagement mechanism, a magnetic engagement mechanism, a thermal engagement mechanism, and an adhesive engagement mechanism, wherein the complementary locking mechanism is configured to fixedly engage the locking mechanism of the medial component distal base side in the assembled configuration of the endoprosthetic knee implant modular tibial insert assembly.

In an exemplary embodiment of the endoprosthetic knee implant modular tibial insert assembly, the medial component proximal side optionally further comprises a medial component articular surface, and wherein the medial component articular surface is a medial pivot medial component articular surface.

In an exemplary embodiment of the endoprosthetic knee implant modular tibial insert assembly, the lateral component proximal side optionally further comprises a lateral component articular surface, and wherein the lateral component articular surface is a medial pivot lateral component articular surface.

In an exemplary embodiment of the endoprosthetic knee implant modular tibial insert assembly, the medial component body optionally further comprises a medial component outer periphery side and a medial component inner periphery side. In such an exemplary embodiment, the lateral component body optionally further comprises a lateral component outer periphery side and a lateral component inner periphery side. In a further such exemplary embodiment, the medial component inner periphery side mechanically engages the lateral component inner periphery side in the assembled configuration.

In an exemplary embodiment of the endoprosthetic knee implant modular tibial insert assembly, the lateral component body optionally further comprises a lateral component outer periphery side and a lateral component inner periphery side.

In an exemplary embodiment of the endoprosthetic knee implant modular tibial insert assembly, the medial component body and the lateral component body optionally further comprise a locking mechanism selected from the group consisting essentially of: a projection-receiver engagement mechanism, a magnetic engagement mechanism, a thermal engagement mechanism, and an adhesive engagement mechanism.

In an exemplary embodiment of the endoprosthetic knee implant modular tibial insert assembly, the modular medial component distal base side is optionally configured to fixedly engage the tibial baseplate.

In an exemplary embodiment of the endoprosthetic knee implant modular tibial insert assembly, the modular medial insert component comprises a biocompatible material selected from the group consisting essentially of: a polyethylene, a polyether ether ketone, and a ceramic.

In an exemplary embodiment of the endoprosthetic knee implant modular tibial insert assembly, the modular lateral insert component comprises a biocompatible material selected from the group consisting essentially of: a polyethylene, a polyether ether ketone, and a ceramic.

In an exemplary embodiment of the endoprosthetic knee implant modular tibial insert assembly, the medial component body further comprises a medial component outer periphery side and a medial component inner periphery side, the lateral component body further comprises a lateral component outer periphery side and a lateral component inner periphery side, wherein the medial component inner periphery side physically abuts the lateral component inner periphery side in the assembled configuration. In one such embodiment, a distal edge of the medial component outer periphery side abuts a medial peripheral edge of a tibial base component, and a distal edge of the lateral component outer periphery side abuts a lateral peripheral edge of the tibial base component in the assembled configuration.

In an exemplary embodiment, the endoprosthetic knee implant modular tibial insert assembly further comprises a plurality of modular medial insert components, the plurality of modular medial insert components comprising a first modular medial insert component and a second modular medial insert component, the first modular medial insert component having a different specification than the second modular medial insert component, wherein the specification is selected from the group consisting essentially of: an AP slope angle, an ML slope angle, a minimum thickness, a size, a dwell point location, an articular surface, a radius of curvature, and a biocompatible material.

In an exemplary embodiment, the endoprosthetic knee implant modular tibial insert assembly further comprises a plurality of modular lateral insert components, the plurality of modular lateral insert components comprising a first modular lateral insert component and a second modular lateral insert component, the first modular lateral insert component having a different specification than the second modular lateral insert component, wherein the specification is selected from the group consisting essentially of: an AP slope angle, an ML slope angle, a minimum thickness, a size, an articular surface, and a biocompatible material.

An exemplary endoprosthetic knee implant modular medial tibial insert component comprises: a medial component proximal side proximally disposed from a medial component distal base side, wherein the medial component distal base side comprises a distal locking mechanism configured to engage the baseplate of a tibial component; a medial component anterior side anteriorly disposed from a medial component posterior side; a medial component outer periphery side medially disposed from a medial component inner periphery side, wherein an anterior edge separates the medial component anterior side from the medial component inner periphery side and a posterior edge separates the medial component posterior side from the medial component inner periphery side; a medial component body extending between the medial component proximal side and the medial component distal base side, the medial component outer periphery side and the medial component inner periphery side, and the medial component anterior side and the medial component posterior side; and an articular surface disposed at the medial component proximal side, wherein the articular surface is a medial pivot articular surface.

In an embodiment of the endoprosthetic knee implant modular medial tibial insert component, the medial component inner periphery side is substantially planar.

In an embodiment of the endoprosthetic knee implant modular medial tibial insert component, the modular medial tibial insert component is configured to mechanically engage a modular lateral tibial insert at the medial component inner periphery.

An exemplary endoprosthetic knee implant modular lateral tibial insert component comprises: a lateral component proximal side proximally disposed from a lateral component distal base side, wherein the lateral component distal base side comprises a distal locking mechanism configured to engage the baseplate of a tibial component; a lateral component anterior side anteriorly disposed from a lateral component posterior side; a lateral component outer periphery side laterally disposed from a lateral component inner periphery side, wherein an anterior edge separates the lateral component anterior side from the lateral component inner periphery side and a posterior edge separates the lateral component posterior side from the lateral component inner periphery side; a lateral component body extending between the lateral component proximal side and the lateral component distal base side, the lateral component outer periphery side and the lateral component inner periphery side, and the lateral component anterior side and the lateral component posterior side; and an articular surface disposed at the lateral component proximal side, wherein the articular surface is a medial pivot articular surface.

In an embodiment of the endoprosthetic knee implant modular lateral tibial insert component, the lateral component inner periphery side is optionally substantially planar.

In an embodiment of the endoprosthetic knee implant modular lateral tibial insert component, the modular lateral tibial insert component is optionally configured to mechanically engage a modular medial tibial insert at the lateral component inner periphery.

An exemplary method of assembling an endoprosthetic knee implant modular tibial insert assembly comprises the following steps: step a: selecting a modular medial tibial insert component from a supply of modular medial tibial insert components to define a selected modular medial tibial insert component, wherein each modular medial insert component in the supply of modular medial insert components has a specification, and wherein the specification of the selected modular medial insert component differs from a specification of another modular medial tibial insert component in the supply of modular medial insert components, the selected modular medial tibial insert component having: a medial component proximal side proximally disposed from a medial component distal base side, wherein the medial component distal base side comprises a medial component distal locking mechanism configured to engage a complementary medial component proximal locking mechanism of a baseplate of a prior implanted tibial component, a medial component outer periphery side medially disposed from a medial component inner periphery side, wherein an anterior edge separates the medial component anterior side from the medial component inner periphery side and a posterior edge separates the medial component posterior side from the medial component inner periphery side, a medial component body extending between the medial component proximal side and the medial component distal base side, the medial component outer periphery side and the medial component inner periphery side, and the medial component anterior side and the medial component posterior side, and a medial component articular surface disposed at the medial component proximal side, wherein the medial component articular surface is a medial pivot articular surface; step b: selecting a modular lateral tibial insert component from a supply of modular lateral tibial insert components to define a selected modular lateral tibial insert component, wherein each modular lateral insert component in the supply of modular lateral insert components has a specification, and wherein the specification of the selected modular lateral insert component differs from a specification of another modular lateral tibial insert component in the supply of modular lateral insert components, the selected modular lateral tibial insert component having: a lateral component proximal side proximally disposed from a lateral component distal base side, wherein the lateral component distal base side comprises a lateral component distal locking mechanism configured to engage a complementary lateral component proximal locking mechanism of the baseplate of the prior implanted tibial component, a lateral component outer periphery side laterally disposed from a lateral component inner periphery side, wherein an anterior edge separates the lateral component anterior side from the lateral component inner periphery side and a posterior edge separates the lateral component posterior side from the lateral component inner periphery side, a lateral component body extending between the lateral component proximal side and the lateral component distal base side, the lateral component outer periphery side and the lateral component inner periphery side, and the lateral component anterior side and the lateral component posterior side, and a lateral component articular surface disposed at the medial component proximal side, wherein the lateral component articular surface is a medial pivot articular surface; step c: placing the selected modular medial tibial insert component onto the baseplate of the prior implanted tibial component, and locking the medial component distal locking mechanism to the complementary proximal medial locking mechanism of the baseplate of the prior implanted tibial component to thereby fixedly engage the selected modular medial tibial insert component to the tibial baseplate; step d: placing the selected modular lateral tibial insert component onto the baseplate of the prior implanted tibial component and locking the lateral component distal locking mechanism to the complementary proximal lateral locking mechanism of the baseplate of the prior implanted tibial component to thereby fixedly engage the selected modular lateral tibial insert component to the tibial baseplate; step e: evaluating a range of motion of a patient's knee joint intraoperatively; and step f: repeating steps a through e until a desired range of motion of the operative knee is achieved.

In an exemplary embodiment, the method further comprises removing the selected modular medial tibial insert component fixedly engaged to the tibial baseplate in step c if the desired range of motion of step f is not achieved. This exemplary embodiment can further comprise: selecting a further modular medial tibial insert component from the supply of modular medial tibial insert components to define a further selected modular medial tibial insert component, the further selected modular medial tibial insert component having: a medial component proximal side proximally disposed from a medial component distal base side, wherein the medial component distal base side comprises a medial component distal locking mechanism configured to engage the complementary medial component proximal locking mechanism of a baseplate of a prior implanted tibial component, a medial component outer periphery side medially disposed from a medial component inner periphery side, wherein an anterior edge separates the medial component anterior side from the medial component inner periphery side and a posterior edge separates the medial component posterior side from the medial component inner periphery side, a medial component body extending between the medial component proximal side and the medial component distal base side, the medial component outer periphery side and the medial component inner periphery side, and the medial component anterior side and the medial component posterior side, and a medial component articular surface disposed at the medial component proximal side, wherein the medial component articular surface is a medial pivot articular surface; placing the further selected modular medial tibial insert component onto the baseplate of the prior implanted tibial component; and locking the medial component distal locking mechanism with the complementary medial component proximal locking mechanism of the baseplate of the prior implanted tibial component to thereby fixedly engage the further selected modular medial tibial insert component to the tibial baseplate.

In an exemplary embodiment, the independent method described above can further comprise: removing the selected modular lateral tibial insert component fixedly engaged to the tibial baseplate in step d if the desired range of motion of step f is not achieved. This particular exemplary embodiment can further comprise: selecting a further modular lateral tibial insert component from the supply of modular lateral tibial insert components to define a further selected modular lateral tibial insert component, the further selected modular lateral tibial insert component having: a lateral component proximal side proximally disposed from a lateral component distal base side, wherein the lateral component distal base side comprises a lateral component distal locking mechanism configured to engage a complementary lateral component proximal locking mechanism of the baseplate of the prior implanted tibial component, a lateral component outer periphery side laterally disposed from a lateral component inner periphery side, wherein an anterior edge separates the lateral component anterior side from the lateral component inner periphery side and a posterior edge separates the lateral component posterior side from the lateral component inner periphery side, a lateral component body extending between the lateral component proximal side and the lateral component distal base side, the lateral component outer periphery side and the lateral component inner periphery side, and the lateral component anterior side and the lateral component posterior side, and a lateral component articular surface disposed at the medial component proximal side, wherein the lateral component articular surface is a medial pivot articular surface; placing the further selected modular lateral tibial insert component onto the baseplate of the prior implanted tibial component; and locking the lateral component distal locking mechanism with the complementary lateral component proximal locking mechanism of the baseplate of the prior implanted tibial component to thereby fixedly engage the further selected modular lateral tibial insert component to the tibial baseplate. Any of the exemplary methods described herein can further comprise fixedly engaging the selected modular medial tibial insert component to the selected modular lateral tibial insert component.

An exemplary modular tibial insert assembly comprises: a modular medial tibial insert component comprising: a medial component proximal side proximally disposed from a medial component distal base side, wherein the medial component distal base side comprises a distal locking mechanism configured to engage the baseplate of a tibial component, a medial component anterior side anteriorly disposed from a medial component posterior side, a medial component outer periphery side medially disposed from a medial component inner periphery side, wherein an anterior edge separates the medial component anterior side from the medial component inner periphery side and a posterior edge separates the medial component posterior side from the medial component inner periphery side, a medial component body extending between the medial component proximal side and the medial component distal base side, the medial component outer periphery side and the medial component inner periphery side, and the medial component anterior side and the medial component posterior side, and an articular surface disposed at the medial component proximal side, wherein the articular surface is a medial pivot articular surface; and a modular lateral tibial insert component comprising: a lateral component proximal side proximally disposed from a lateral component distal base side, wherein the lateral component distal base side comprises a distal locking mechanism configured to engage the baseplate of a tibial component, a lateral component anterior side anteriorly disposed from a lateral component posterior side, a lateral component outer periphery side laterally disposed from a lateral component inner periphery side, wherein an anterior edge separates the lateral component anterior side from the lateral component inner periphery side and a posterior edge separates the lateral component posterior side from the lateral component inner periphery side, a lateral component body extending between the lateral component proximal side and the lateral component distal base side, the lateral component outer periphery side and the lateral component inner periphery side, and the lateral component anterior side and the lateral component posterior side, and an articular surface disposed at the lateral component proximal side, wherein the articular surface is a medial pivot articular surface, and wherein the modular tibial insert assembly has an assembled configuration and a disassembled configuration.

In at least one such an exemplary embodiment, the modular medial tibial insert component is a first tibial insert component, the modular lateral tibial insert component is a second tibial insert component and the modular tibial insert assembly further comprises a third tibial insert component, wherein the third tibial insert is configured to be disposed between the first tibial insert component and the second tibial insert component in the assembled configuration. In at least one such exemplary embodiment, the third tibial insert component is configured to fixedly engage the first tibial insert component or the second tibial insert component in the assembled configuration. In at least one such exemplary embodiment, the third tibial insert component is configured to fixedly engage the first tibial insert component and the second tibial insert component in the assembled configuration. In at least one such exemplary embodiment, the third tibial insert component is a constrained condylar knee modular tibial insert component. In at least one other such exemplary embodiment, the third tibial insert component is a posterior stabilized knee modular tibial insert component.

In at least one exemplary embodiment comprising a third tibial insert component, the modular tibial insert assembly further comprises a fourth tibial insert component, wherein the fourth tibial insert component is a modular stabilizing post configured to be disposed between the third tibial insert component and a baseplate of a tibial component in an assembled and implanted configuration.

Although the present invention has been described in terms of specific embodiments, it is anticipated that alterations and modifications thereof will no doubt become apparent to those skilled in the art. It is therefore intended that the following claims be interpreted as covering all alterations and modifications that fall within the true spirit and scope of the invention.

What is claimed is:

1. A modular tibial insert assembly comprising:
a modular medial insert component, the modular medial insert component having a medial component proximal side proximally disposed from a medial component distal base side, wherein a medial component body extends between the medial component proximal side and the medial component distal base side;
a modular lateral insert component, the modular lateral insert component having a lateral component proximal side proximally disposed from a lateral component distal base side, wherein a lateral component body extends between the lateral component proximal side and the lateral component distal base side;
wherein the modular tibial insert assembly has an assembled configuration and a disassembled configuration, and
wherein the modular medial insert component is configured to fixedly engage the modular lateral insert component in the assembled configuration.

2. The modular tibial insert assembly of claim 1, wherein the medial component proximal side further comprises a medial component articular surface, and wherein the medial component articular surface is a medial pivot medial component articular surface.

3. The modular tibial insert assembly of claim 1, wherein the lateral component proximal side further comprises a lateral component articular surface, and wherein the lateral component articular surface is a medial pivot lateral component articular surface.

4. The modular tibial insert assembly of claim 1, wherein the medial component body further comprises a medial component outer periphery side and a medial component inner periphery side.

5. The modular tibial insert assembly of claim 4, wherein the lateral component body further comprises a lateral component outer periphery side and a lateral component inner periphery side.

6. The modular tibial insert assembly of claim 5, wherein the medial component inner periphery side mechanically engages the lateral component inner periphery side in the assembled configuration.

7. The modular tibial insert assembly of claim 1, wherein the lateral component body further comprises a lateral component outer periphery side and a lateral component inner periphery side.

8. The modular tibial insert assembly of claim 1, wherein the medial component body and the lateral component body further comprise a locking mechanism selected from the group consisting essentially of: a projection-receiver engagement mechanism, a magnetic engagement mechanism, a thermal engagement mechanism, and an adhesive engagement mechanism.

9. The modular tibial insert assembly of claim 1 further comprising a plurality of modular medial insert components, the plurality of modular medial insert components comprising a first modular medial insert component and a second modular medial insert component, the first modular medial insert component being of a different size than the second modular medial insert component, wherein the modular medial insert component is selected from the plurality of modular medial insert components.

10. The modular tibial insert assembly of claim 1 further comprising a plurality of modular lateral insert components, the plurality of modular lateral insert components comprising a first modular lateral insert component and a second modular lateral insert component, the first modular lateral insert component being of a different size than the second modular lateral insert component, wherein the modular lateral insert component is selected from the plurality of modular lateral insert components.

11. The modular tibial insert assembly of claim 1 further comprising a tibial base component, the tibial base component comprising a tibial baseplate.

12. The modular tibial insert assembly of claim 11, wherein the modular medial component distal base side is configured to fixedly engage the tibial baseplate.

13. The modular tibial insert assembly of claim 11, wherein the modular lateral component distal base side is configured to fixedly engage the tibial baseplate.

14. The modular tibial insert assembly of claim 1, wherein the modular medial insert component comprises a biocompatible material selected from the group consisting essentially of: a polyethylene, a polyether ether ketone, and a ceramic.

15. The modular tibial insert assembly of claim 1, wherein the modular lateral insert component comprises a biocompatible material selected from the group consisting essentially of: a polyethylene, a polyether ether ketone, and a ceramic.

16. The modular tibial insert assembly of claim 1, wherein the medial component body further comprises a medial component outer periphery side and a medial component inner periphery side, the lateral component body further comprises a lateral component outer periphery side and a lateral component inner periphery side, wherein the medial component inner periphery side physically abuts the lateral component inner periphery side in the assembled configuration.

17. The modular tibial insert assembly of claim 16, wherein a distal edge of the medial component outer periphery side abuts a medial peripheral edge of a tibial base component, and wherein a distal edge of the lateral component outer periphery side abuts a lateral peripheral edge of the tibial base component in the assembled configuration.

18. The modular tibial insert assembly of claim 1 further comprising a plurality of modular medial insert components, the plurality of modular medial insert components comprising a first modular medial insert component and a second modular medial insert component, the first modular medial insert component having a different specification than the second modular medial insert component, wherein the specification is selected from the group consisting essentially of: an AP slope angle, an ML slope angle, a minimum thickness, a dwell point location, an articular surface, a radius of curvature, and a biocompatible material.

19. The modular tibial insert assembly of claim 1 further comprising a plurality of modular lateral insert components, the plurality of modular lateral insert components comprising a first modular lateral insert component and a second modular lateral insert component, the first modular lateral insert component having a different specification than the second modular lateral insert component, wherein the specification is selected from the group consisting essentially of: an AP slope angle, an ML slope angle, a minimum thickness, an articular surface, and a biocompatible material.

20. An endoprosthetic knee implant modular tibial insert assembly comprising:
a modular medial tibial insert component, the modular medial tibial insert component having a medial component proximal side proximally disposed from a medial component distal base side, wherein a medial component body extends between the medial component proximal side and the medial component distal base side;
a modular lateral tibial insert component, the modular lateral tibial insert component having a lateral component proximal side proximally disposed from a lateral component distal base side, wherein a lateral component body extends between the lateral component proximal side and the lateral component distal base side;
a tibial component comprising a tibial baseplate, the tibial baseplate comprising a superior surface;
wherein the endoprosthetic knee implant modular tibial insert assembly has an assembled configuration and a disassembled configuration, and
wherein the modular medial tibial insert component is configured to fixedly engage the tibial component in the assembled configuration.
